(19) United States
(12) Patent Application Publication
(10) Pub. No.: US 2018/0172683 A1
(43) Pub. Date: Jun. 21, 2018

(54) METHODS FOR ANALYZING THE INTERACTION BETWEEN A TARGET PROTEIN AND A LIGAND

(71) Applicant: UNIVERSITY OF SOUTHERN CALIFORNIA, Los Angeles, CA (US)

(72) Inventors: Richard ROBERTS, South Pasadena, CA (US); Farzad JALALI-YAZDI, Mission Viejo, CA (US)

(21) Appl. No.: 15/738,366

(22) PCT Filed: Jun. 22, 2016
(86) PCT No.: PCT/US2016/038830

§ 371 Date: Dec. 20, 2017

Related U.S. Application Data
(60) Provisional application No. 62/183,111, filed on Jun. 22, 2015.
(60) Provisional application No. 62/183,113, filed on Jun. 22, 2015.

(51) Int. Cl. G01N 33/557; G06F 19/20; G01N 33/53; G01N 33/543; G01N 33/566; C12Q 1/6804

(52) U.S. Cl. CPC G01N 33/557; G06F 19/20; G01N 33/5306; G01N 2800/7028; G01N 33/54326; G01N 33/566; C12Q 1/6804; G01N 33/5302

(57) ABSTRACT

Provided is a method for simultaneously determining both [L]₀ and Kd values of a ligand for a target protein. In one embodiment, the present technology involves performing quantitative equilibrium immunoassays at two different concentrations of the target and fitting the data to simultaneously determine K_{d} and [L]₀. Also provided is a method for determining binding affinity of a pool of candidate ligands in a high-throughput manner. In another embodiment, the present technology method combines high-throughput nucleic acid sequencing with a display technology to obtain kinetic on-rates and off-rates, and thus K_{d} values, for the candidate ligands.

### CROSS-REFERENCE TO RELATED APPLICATION(S)

This application claims priority to U.S. Provisional Application No. 62/183,111, filed on Jun. 22, 2015, and U.S. Provisional Application No. 62/183,113, filed on Jun. 22, 2015, the entire contents of all of which are incorporated herein by reference.

### GOVERNMENT RIGHTS NOTICE

This invention was made with government support under grant numbers R01AI085583 and R01CA170820, awarded by National Institute of Health (NIH). The government has certain rights in the invention.

### FIELD

The present technology generally relates to the measurement of the interaction between a target protein and a ligand. In particular, the present technology relates to a method for determining the dissociation constant (K_{d}) and ligand concentration ([L]₀) simultaneously using a direct, label-free, and general approach. The present technology also relates to a method for evaluating the affinity of a pool of candidate ligands against a target protein in a high-throughput manner.

### BACKGROUND

Immune assays remain the most widely used method for protein detection, tracking, and characterization. The generation of proteome-wide immune reagents provides an important route to address cancer biology, immunology, and basic research. However, a problem with quantitative analysis using antibody-based assays is that neither the antibody concentration ([L]₀) nor the K_{d} for the target are generally known. This is suboptimal in a variety of important situations ranging from antibody screening to quantitative immunoassays, and in the development of therapeutic antibodies where efficacy directly relates to affinity and specificity. A second issue with antibody-based diagnostics is that the prevailing model for analyzing equilibrium data treats antibodies as monovalent reagents. A third major issue is that measuring K_{d} for high affinity ligands can be challenging because long off-rates can bias results, while some indirect methods require chemical labeling of ligands, which can alter K_{d}. Accordingly, there is a need for new immune assays that address these challenges.

Various in vitro selection techniques (such as phage display, ribosome display, and mRNA display) have facilitated the generation of polypeptide ligands against targets of interest. The challenge, increasingly, is ranking the molecules based on their desirable properties, including their affinity for their targets. For example, although it has been shown that sequences with higher copies in a pool after selection do exhibit functionality, a sequence's rank does not necessarily correlate with its absolute fitness. Specifically, a higher ranked sequence does not always have higher affinity to the target than a lower ranked one. Thus, there is a need for characterizing ligand affinity by an ultra-high-throughput method. Advances in the field have been able to increase the throughput of K_{d} measurements using radioactivity, SPR or fluorescent microarrays, and ELISA assays. However, all of these methods require individually expressed and purified ligands, greatly reducing their throughput. Measuring the K_{d} for thousands of potential ligands simultaneously has not yet been realized.

### SUMMARY

In one aspect, the present technology provides a method for simultaneously determining [L]₀ and K_{d} of a ligand for a target protein, which includes the steps of: (1) conducting a first quantitative equilibrium immunoassay of the ligand with the target protein at a first concentration of the target protein; (2) conducting a second quantitative equilibrium immunoassay of the ligand with the target protein at a second concentration of the target protein; and (3) fitting the data resulting from steps (1) and (2) to determine K_{d} and [L]₀ simultaneously. In some embodiments, the present method includes a forward immunoassay, in which the ligand is immobilized and the target protein is in solution. In other embodiments, the present method includes a reverse immunoassay, in which the target protein is immobilized and the ligand is in solution. Further, the fitting step of the present method can use either a monovalent model or a divalent model for the binding between the target protein and the ligand.

In another aspect, the present technology provides a method for determining binding affinity, the method comprising: (1) preparing a pool of candidate ligands; (2) mixing the pool of candidate ligands with a target protein immobilized on a carrier; (3) isolating the mixture of step (2); (4) sequencing the candidate ligands bound to the target protein to identify a pool of nucleic acid sequences; (5) translating each of the nucleic acid sequences in the pool of sequences identified in step (4); and (6) calculating a frequency of each translated sequence generated in step (5). In one embodiment, the candidate ligands include mRNA-peptide fusion molecules. In another embodiment, the target protein is B-cell lymphoma extra-large protein (Bcl-xL) immobilized on magnetic beads. The present method can be used to evaluate the affinity of candidate ligands against a target protein in a high-throughput manner. The present method can also include the step of calculating the kinetic on-rate or off-rate for each candidate ligand sequence.

Other aspects of the invention will become apparent by consideration of the detailed description and accompanying drawings.

### BRIEF DESCRIPTION OF THE DRAWINGS

FIG. 1 shows the measurement of K_{d} via forward equilibrium immunoassays (target in solution). (a) Schematic for generating ELISA signal: Target protein (Bcl-xL) binds capture-ligand immobilized on the ELISA plate. Bound target is quantified with a detection-antibody/HRP conjugate. Target equilibrated with increasing concentrations of a competing ligand (here Bim) reduces the signal, since the pre-formed ligand/target complex cannot interact with the ELISA plate. (b) ELISA signal for known concentrations of Bcl-xL fit to a 4 parameter logistic model. Two target concentrations (1 nM and 111 pM) were chosen for pre-incubation with Bim. (c) Loss of ELISA signal resulting from equilibrating 1 nM or 111 pM Bcl-xL (red diamonds and squares, respectively) with Bim. The signal represents the unbound target (concentration calculated using panel (b)). (d) Determining the K_{d} value for the ligand. The fraction of Bcl-x_{L} bound (% C_{EQ}, diamonds and squares) and ligand concentrations are fit to the equilibrium model (FIGS. 6 and 7). Data from both high and low target concentrations are fit simultaneously to obtain a K_{d} value. (e) Schematic for K_{d} measurements generated via AMMP. The capture-ligand is immobilized on magnetic beads and incubated with target and fluoresceinated detection-antibody. Binding of the detection-antibody to the anti-fluorescein antibody on the sensor surface connects the magnetic bead to the sensor, generating signal. As with the ELISA assay, signal is reduced when a competing ligand is equilibrated with the target. (f) The K_{d} values obtained using the AMMP assay are equivalent to the results obtained by ELISA for peptides, small molecule and antibody ligands.

FIG. 2 shows simultaneous fitting of K_{d} and [L]₀ produces accurate results. (a) Fitting for K_{d} and [L]₀ simultaneously yields K_{d} values that are equivalent to the values obtained when [L]₀ is known. (b) Ligand concentrations determined by simultaneous fitting of K_{d} and [L]₀ match the known [L]₀. (c) Simultaneous fitting of K_{d} and [L]₀ for peptide E1 using the monovalent equilibrium model yields a unique solution (red line). Light grey and dark gray dashed lines demonstrate the fidelity of the fit to the high (T_{H}, red diamonds) and low (T_{L}, red squares) target concentration samples when K_{d} and [L]₀ are each varied ±10-fold while the other variable is held constant. Here, the x-axis is given as relative concentration (DF⁻¹) since [L]₀ is unknown. (d) 3-D surface plot showing the error (absolute deviation, z-axis) between a simulated data set calculated from true [L]₀ and K_{d} values, and data sets where [L]₀ and K_{d} are allowed to vary ±100-fold from their true values. A unique and accurate solution for [L]₀ and K_{d} can be determined if the error surface only approaches the x-y plane at the true values of [L]₀ and K_{d}. (e) and (0 The lowest values of the projected error surface as viewed on the error vs [L]₀ or error vs K_{d} planes, respectively (details in FIG. 9). A higher error projection (e.g., the blue projection in panel (c) corresponds to higher sensitivity of the measured parameter resulting in better accuracy and precision.

FIG. 3 shows fitting using two or more target concentrations that bracket K_{d} is required to derive accurate values for K_{d} and [L]₀. The above data points were simulated to illustrate the range where simultaneously fitting for K_{d} and [L]₀ produce accurate results. For each plot, the fit K_{d} value was set to 5-fold the true K_{d} value, and the fit [L]₀ value was chosen to minimize the error. The data points and the black lines represent the true K_{d} and [L]₀ values for each plot. (a) Within the optimal range for accurate K_{d} and [L]₀ measurement by simultaneous fitting (T_{H}>K_{d}>0.1×T_{L}, obtained from FIGS. 2e and 20, the erroneously fit K_{d} and [L]₀ (red dashed lines) do not match the data. However, when using a single target concentration (panel (b)) or working outside the appropriate target concentration ranges (panels (c) and (d)), plots using the erroneous values (red dashed lines) can show good overlap with the data, despite a five-fold deviation in K_{d}.

FIG. 4 shows that, in the forward assay, accurate K_{d} and [L]₀ values can be determined by modeling antibodies as monovalently bound ligands. (a) Schematic to generate the standard curve for the forward assay. Target protein (Bcl-xL) binds to an immobilized antibody a solid support (here, ELISA plate) in monovalent or divalent format. Bound target is quantified with a detection-antibody/HRP conjugate. (b) Schematic for the forward assay at equilibrium. Equilibration of target and antibody generates both monovalently-bound and divalently-bound target-ligand complexes. Neither complex can interact with the immobilized antibody on the solid support, lowering the signal similarly to FIG. 1c. (c) The traditional approach to determine binding constants (a monovalent model using the number of antibody sites as the ligand concentration) results in both large errors and erroneous K_{d} values (dashed black lines) when fit for both target concentrations. A model treating the ligand as divalent results in better fits at both target concentrations (red lines). (d) Simultaneous fitting of K_{d} and [L]₀ results in excellent fits for both monovalent and divalent models and gives identical values for K_{d}, but results in a two-fold difference in the fit ligand concentration (R_{L}=the ratio of the fit [L]₀ to known [L]₀). The K_{d} values from the simultaneous fits also match well with the divalent K_{d} only fits in panel (c). (e) Fraction of signal due to monovalent (red dashes) and divalent (red dots) antibody-target complexes. In the forward assay, >99% of the signal arises from the monovalent complex.

FIG. 5 shows that, in the reverse assay (target immobilized), determining K_{d} and [L]₀ can only be done accurately when a divalent model is used. (a) Schematic to generate the standard curve for the reverse assay. The antibody can bind to a single immobilized target on solid support or it can bridge two nearby target proteins. Bound antibody is quantified with a detection-antibody/HRP conjugate. (b) Schematic for the reverse assay at equilibrium. The monovalently bound ligand can bind to the immobilized target and give rise to signal whereas the divalently bound ligand cannot. (c) Calculating the K_{d} values for the reverse immunoassays. The best-fit curve of the monovalent equilibrium model does not match the experimental data for either high (blue diamonds) or low (blue squares) ligand concentration sets. In contrast, the divalent model (solid line) matches the data very closely. (d) Simultaneous fitting of K_{d} and [L]₀ for the reverse assay. The monovalent model does not match the data when K_{d} and [L]₀ are fit simultaneously. Both the divalent and the monovalent K_{d} values are similar to the calculated values in panel (c). (e) The divalent complex has a very significant contribution in the reverse assay. At low target concentrations, the monovalent complex dominates the signal, whereas at high target concentrations, the divalent complex has a greater contribution. This effect can be treated using a negative cooperativity term (C_{f}) corresponding to the percent of monovalently bound ligand that does not interact with the immobilized target.

FIG. 6 shows the monovalent model and formulas governing the equilibrium and transient behavior of a simple binary binding system.

FIG. 7 shows the divalent model and formulas governing the equilibrium behavior of divalent ligand.

FIG. 8 shows that iterative fitting methods can produce stable but erroneous pairs of K_{d} and [L]₀ values. Panels a and b show the calculated error using true K_{d} and [L]₀ vs the iterative optimization method developed by Darling and Brault (red). Sequential optimization can result in stable pairs for the fit K_{d} and fit [L]₀ that minimize the calculated error, but do not match the true K_{d} and [L]₀. Plotting the target bound vs. dilution factor for the example in panel (c), demonstrates that the true K_{d} and [L]₀ values accurately fit all the data (black lines), whereas the sequential method (red dashed lines) does not.

FIG. 9 shows obtaining lowest error values as a tool for assessing parameter sensitivity. (a) Deviation between the true % C_{EQ} and % C_{EQ} obtained by varying K_{d} and [L]₀ each by 2 orders of magnitude (error) where T_{H}=true K_{d}. (b) Projection of panel (a) on the [L]₀ vs. error plane. (c) Projection of panel (a) on the K_{d} vs. error plane. (d) The lowest error obtained from panels (b) and (c). The lowest error for a given [L]₀ deviation on the graph to the left provides the minimum error generated by testing all K_{d} values.

FIG. 10 shows that using a single target concentration leads to underdetermined K_{d} and [L]₀ values. (a) and (b) Minimum values for the 3D-error surface as viewed on the [L]₀ vs. error plane or K_{d} vs. error plane, respectively (details of this process are shown in FIG. 7). The error projections are much broader than when two concentration of target are used (FIGS. 2c and 2d) making it difficult to uniquely determine accurate values for K_{d} and [L]₀, since there are multiple values of K_{d} or [L]₀, that result in small minimum errors. A single target concentration thus results in lower precision and accuracy of the fit K_{d} and [L]₀, values.

FIG. 11 shows the advantages of the AMMP assay over ELISA. (a) AMMP assay signal for Bcl-xL Standards is fit to a 4 parameter logistic model. The magnetic beads collected on the AMMP sensor surface are washed at three flow rates: low (blue circles, highest sensitivity), medium (red diamonds) and high (black squares). The use of the three flow rates extends the dynamic range of the assay to ˜3 log units. (b) The AMMP assay is more sensitive than ELISA for identical samples and affinity reagents. The Lower Limit of Quantification (LLOQ) for the assays are marked with a green arrow (ELISA, 37 pM) and a blue arrow (AMMP 4 pM).

FIG. 12 shows the kinetic rates for ligands obtained by using high-throughput sequencing kinetic (HTSK). FIG. 12a show the results in obtaining the kinetic on-rate. The pool of mRNA-peptide fusion molecules was incubated with Bcl-xL (immobilized on beads). At specific time points, a fraction of beads were collected and washed. The molecules bound to the beads were sequenced via next-generation sequencing. The fraction of each ligand at each time point was calculated from the sequencing data and normalized with respect to the final data point (left). Separately, the pool was in vitro translated using radiolabeled methionine, and its binding was determined at each time point (middle). The ligand's contribution to the radiolabeled binding and, subsequently, the on-rate (right) were obtained by multiplying each ligand's composition fraction by the radiolabeled binding at each data point. FIG. 12b shows the results in obtaining the kinetic off-rate. At the end of the on-rate experiment, the remaining beads were washed and placed in a solution containing 100× excess Bcl-xL in solution, preventing ligands from re-binding to the beads. At specific time points, a fraction of beads were collected and washed. The molecules still bound to the beads were analyzed by next-generation sequencing. The fraction of each ligand at each time point was calculated from the sequencing data and normalized with respect to the first data point (left). The counts remaining on the beads at each time point were measured using the radiolabeled sample (middle). By multiplying each ligand's composition fraction by the radiolabeled binding at each data point, the ligand's contribution to the radiolabeled binding and the off-rate were obtained (right).

FIG. 13 shows that the HTSK results are reproducible and accurate. FIG. 13a shows the obtained K_{d} for the top 50 clones in the extension and doped pools. While the extension pool on average (dashed red line) is comprised of lower affinity binder than the doped pool (dashed blue lines), some sequences in the extension pool show higher affinity than the doped pool average. FIG. 13b shows the obtained HTSK values are reproducible. 40 sequences appeared in both the extension and the doped pools. Comparing the kinetic constants for these sequences shows that the results are reproducible. FIG. 13c shows the k_{off} value obtained by HTSK correlate well to the values obtained using radiolabeled peptides. There is a consistent bias in the measured off-rate values for the two methods of measurements. FIG. 13d shows the radiolabeled peptide off-rate for the previously identified sequences E1 and D1, and the HTSK identified sequence D79. The off-rate for sequence D79 is over 3 times slower than the off-rate of D1, the previously identified highest affinity binder. The slowest reported value for the off-rate of biotin and streptavidin in the literature (2.4×10⁻⁶, Piran et al., Journal of immunological methods, 133, 141-143 (1990)) is shown as a reference.

FIG. 14 shows that ligand E1452 (green circles, frequency rank of 1452 in the extension selection pool) was identified by HTSK and tested as a radiolabeled peptide. Its off-rate is slower than D1, the previously identified highest affinity peptide from the doped selection.

FIG. 15 shows the histogram of the obtained K_{d} values for the extension and doped pools.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

The use of “including,” “comprising,” or “having” and variations thereof herein is meant to encompass the items listed thereafter and equivalents thereof as well as additional items. Any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The present technology relates to a method to determine both K_{d} and [L]₀ values simultaneously by fitting the data to an equilibrium model. The present technology also relates to a method for determining ligand affinity properties, including kinetic on-rates and off-rates and K_{d} values, in a high-throughput manner.

### Definitions

“Antibody” as used herein refers to a human antibody, an immunoglobulin molecule, a disulfide linked Fv, a monoclonal antibody, an affinity matured, a scFv, a chimeric antibody, a single domain antibody, a CDR-grafted antibody, a diabody, a humanized antibody, a multispecific antibody, a Fab, a dual specific antibody, a DVD, a TVD, a Fab′, a bispecific antibody, a F(ab′)2, or a Fv. The antibody may be humanized. The antibody may comprise a heavy chain immunoglobulin constant domain such as, for example, a human IgM constant domain, a human IgG4 constant domain, a human IgG1 constant domain, a human IgE constant domain, a human IgG2 constant domain, a human igG3 constant domain, or a human IgA constant domain.

The term “association rate constant,” “kinetic on-rate”, “on-rate”, or “kₒₙ” as used interchangeably herein, refers to the value indicating the binding rate of a ligand to its target protein or the rate of complex formation between a ligand and protein.

The term “dissociation rate constant,” “kinetic off-rate”, “off-rate”, or “k_{off}” as used interchangeably herein, refers to the value indicating the dissociation rate of a ligand from its target protein or separation of the ligand and protein complex over time into free ligand and free protein.

The term “equilibrium dissociation constant”, “K_{d}”, or “K_{D}” as used interchangeably, herein, refers to the value obtained by dividing the dissociation rate (k_{off}) by the association rate (kₒₙ). The association rate, the dissociation rate and the equilibrium dissociation constant are used to represent the binding affinity of a ligand to a protein.

As used herein, an “immunoassay” means any assay in which the binding of a ligand to a target protein is characterized. The immunoassays may include heterogeneous immunoassays, which involve multiple steps and separation of reagents, and homogenous immunoassays, which do not involve separation of reagents. For example, a homogeneous immunoassay may be carried out by mixing the target protein and the ligand in a solution and subsequently making a physical measurement, such as light absorbance and radiolabel measurements. The immunoassays may be conducted in a competitive or noncompetitive manner. In a competitive immunoassay, two or more different ligands (or target proteins) compete for the binding to the target protein (or the ligand). In a noncompetitive immunoassay, one or more ligands (or target proteins) bind to the target protein (or the ligand) without competition for the binding sites. Non-limiting examples of suitable immunoassay technologies include sandwich immunoassay (e.g., monoclonal-polyclonal sandwich immunoassays, including radioisotope detection (radioimmunoassay (RIA)), enzyme detection (enzyme immunoassay (EIA) or enzyme-linked immunosorbent assay (ELISA) (e.g., Quantikine ELISA assays, R&D Systems, Minneapolis, Minn.)), Acoustic Membrane MicroParticle (AMMP), chemiluminescent microparticle immunoassay (such as one employing the ARCHITECT® automated analyzer, Abbott Laboratories, Abbott Park, Ill.), mass spectrometry, immunohistochemistry, and exclusion immunoassay. An exclusion immunoassay may refer to an immunoassay in which a target protein and a test ligand (the K_{d} of which is being measured) are allowed to reach equilibrium in a medium (such a solution). The sample containing the target protein and the test ligand at equilibrium is added to a substrate containing a capture ligand, which immobilizes the free target protein from the medium to a substrate (such as an ELISA plate). The amount of free target protein immobilized to the substrate is quantitated. In this process, the target protein in complex with the test ligand cannot bind to the capture ligand, and is thus “excluded” from the immunoassay. The same assay also can be carried out using any other immunoassay technologies. For example, the target protein and the test ligand complex may be added to beads with immobilized capture-ligand, which binds the free target protein in solution. The amount of target protein bound to the beads can be quantitated. Other immunoassay technologies known in the art may also be used in the present method.

The term “ligand” as used herein refers to an entity capable of binding to the target protein. The ligand may be a capture ligand which binds to the target protein. The capture-ligand may immobilize the target protein on a solid support. Capture-ligands include, but are not limited to, synthetic peptides suitable for ELISA assays. The ligand may be a competing ligand which competes with the capture ligand to bind the target protein.

The term “sample” as used herein includes protein preparations, cell extracts or lysates, and biological samples such as blood, tissue, urine, serum, plasma, amniotic fluid, cerebrospinal fluid, placental cells or tissue, endothelial cells, leukocytes, or monocytes. The sample can be used directly as obtained from cell culture, animal, or patient, or can be pre-treated, such as by filtration, distillation, extraction, concentration, centrifugation, inactivation of interfering components, addition of reagents, and the like, to modify the character of the sample in some manner as discussed herein or otherwise as is known in the art.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; however, in the event of any latent ambiguity, definitions provided herein take precedent over any dictionary or extrinsic definition. Further, unless otherwise required by context, singular terms shall include pluralities and plural terms shall include the singular.

I. Simultaneous Determination of Dissociation Constant (K_{d}) and Ligand Concentration ([L]₀)

In a first aspect, the present disclosure provides a method for simultaneously determining [L]₀ and K_{d} of a ligand for a target protein. The method includes the steps of:
(1) conducting a first quantitative equilibrium immunoassay of the ligand with the target protein at a first concentration of the target protein;
(2) conducting a second quantitative equilibrium immunoassay of the ligand with the target protein at a second concentration of the target protein; and
(3) fitting the data resulting from steps (1) and (2) to determine K_{d} and [L]₀ simultaneously.

The target protein can be any protein. The target protein may have a ligand binding site and may be suitable for kinetic binding studies. For example, the target protein may be a B-cell Lymphoma extra-large protein (Bcl-xL). The Bcl-xL may be an oncogenic protein that is up-regulated in several types of human carcinomas and a target for therapeutic development.

Suitable ligands for use in the method include, but are not limited to, an antibody, a peptide, or a small molecule compound. In some embodiments, the ligand is an antibody or a peptide. In a particular embodiment, the target protein is Bcl-xL, and the ligand is an antibody, a peptide, or a small molecule compound that binds to Bcl-xL. Suitable antibodies for Bcl-xL include, but are not limited to, commercial monoclonal antibodies (such as 54H6), small molecule compounds (such as the commercial high affinity compound ABT-737), and synthetic peptides. In one embodiment, the ligand is a monoclonal antibody against Bcl-xL.

The “quantitative equilibrium immunoassay” as used herein includes incubating the ligand and the target protein to equilibrium. As a non-limiting example, the target protein can first be incubated with a capture ligand and the amount of free protein quantified. Using different target concentrations, a calibration curve can be generated in order to quantify the amount of free target in solution. To find the K_{d} of samples, the solution containing known amounts of target can be incubated with a ligand (of unknown K_{d}) that competes with the capture ligand. This solution is allowed to equilibrate, reducing the amount of free target protein in solution. The K_{d} of interaction between the target protein and the competing ligand can then be determined by quantifying the amount of free target protein. Any quantitative immunoassay technology capable of sensitive measurement of analyte concentration can be employed for the present method. Suitable quantitative immunoassay technologies include, but are not limited to Enzyme-linked Immunosorbent Assay (ELISA) and Acoustic Membrane MicroParticle (AMMP). Depending on the choice of immunoassay technology, the K_{d} measurement of the present method can reach nanomolar, picomolar, or even sub-picomolar levels. In one embodiment the quantitative equilibrium immunoassay may be a quantitative equilibrium exclusion immunoassay.

The method may include the use of a forward immunoassay, in which the ligand is immobilized and the target protein is in solution. In other embodiments, the method may include a reverse immunoassay, in which the target protein is immobilized and the ligand is in solution. In a forward or reverse immunoassay, the target protein or the ligand may be immobilized on any suitable substrate. As a non-limiting example, the target protein may be immobilized by a capture-ligand on an ELISA plate. As another non-limiting example, the target protein may be immobilized by magnetic beads. The forward assay may especially be useful for screening multiple ligands to find the best binding sequences that can block a specific interaction (e.g., generating therapeutic monoclonal antibodies), as it can rapidly determine the dissociation constants of multiple competing ligands for a single target. If all ligands bind to the same epitope, only a single capture ligand may be needed to create a target response curve, greatly reducing the number of samples needed to accurately measure K_{d} for all ligands. This feature can be used to measure the K_{d} of multiple ligands with a single capture ligand and corresponding standard curve.

The fitting step may include a process of constructing a curve (or mathematical function) according to a specific target-ligand binding model that has the best fit to a series of data points. The target-ligand binding models includes equilibrium models and on- and off-rates equations such as those described herein below, as well as those defined by known equations such as the Hill equation and models for cooperative binding (the Adair equation, the Klotz equation, the Pauling equation, the KNF model, the MWC model, etc.). In one embodiment, the target-ligand binding model includes an equilibrium model, from which the binding constant, the concentration of free unbound ligand, and the concentration of the target-ligand complex may be determined. In the equilibrium model, the binding rate of the ligand to the target protein is balanced by the dissociation constant of the target-ligand complex.

Any data fitting software or tools may be used in the present method for the data fitting step. Non-limiting examples of suitable data fitting software include Excel Solver and MATLAB's fminsearch function.

The method may determine K_{d} and [L]₀ simultaneously, and thus can be used when the concentration of the ligand is known or unknown. The method may be carried out wherein the concentration of the ligand is unknown. Non-limiting examples of samples for which the concentration of the ligand may be unknown include crude, unpurified, partially purified, or purified biological samples, such as tissue samples and cell extracts. In one embodiment, the present method determines K_{d} and [L]₀ simultaneously for one or more ligands.

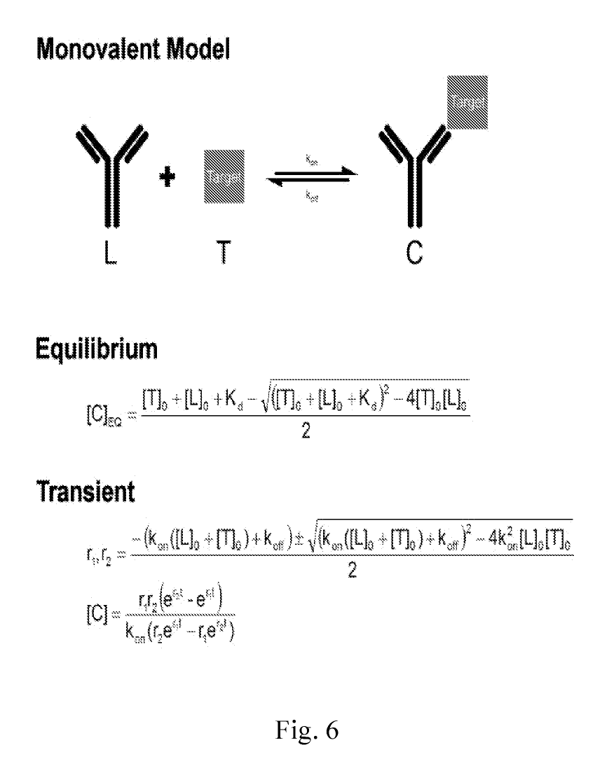

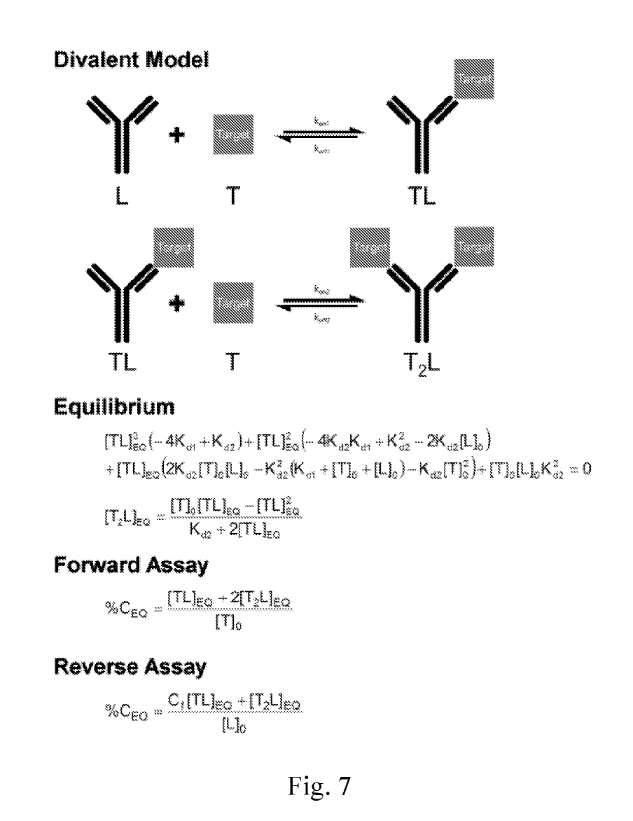

The fitting step of the forward assay method can use either a monovalent model or a divalent model for the binding between the target protein and the ligand. The fitting step of the reverse assay method can use either a monovalent model or a divalent model for the binding between the target protein and the ligand. In a monovalent model, for example, one ligand molecule may interact with one target protein molecule to form a monovalently bound target-ligand complex (TL), in which the molar ratio of the target protein to the ligand is 1:1 (FIG. 6, complex formed by one ligand and one target). In a divalent model, for example, one ligand molecule may interact with two target protein molecules to form a divalently bound target-ligand complex (T₂L), in which the molar ratio of the target protein to the ligand is 2:1 (FIG. 7, complex formed by one ligand and two targets).

In one embodiment, the fitting step may be conducted according to a monovalent model for the binding between the target protein and the ligand. As a non-limiting example, the monovalent model may be:

$${[ C ]}_{EQ} = \frac{{[ T ]}_{0} + {[ L ]}_{0} + {K}_{D} - \sqrt{{( {[ T ]}_{0} + {[ L ]}_{0} + {K}_{D} )}^{2} - {{4 [ T ]}_{0} [ L ]}_{0}}}{2}$$

in which [C]_{EQ} represents the concentration of the target-ligand complex at equilibrium; [T]₀ represents the initial concentration of the target protein; and [L]₀ represents the initial concentration of the ligand (FIG. 6). Other monovalent models known in the art may also be used in the present method.

In another embodiment, the fitting step may be conducted according to a divalent model for the binding between the target protein and the ligand. As a non-limiting example, the divalent model may be:

$${[ TL ]}_{EQ}^{3} ( - 4 {K}_{d 1} + {K}_{d 2} ) + {[ TL ]}_{EQ}^{2} ( - 4 {K}_{d 2} {K}_{d 1} + {K}_{d 2}^{2} - 2 {{K}_{d 2} [ L ]}_{0} ) + {[ TL ]}_{EQ} ( 2 {{{K}_{d 2} [ T ]}_{0} [ L ]}_{0} - {K}_{d 2}^{2} ( {K}_{d 1} + {[ T ]}_{0} + {[ L ]}_{0} ) - {{K}_{d 2} [ T ]}_{0}^{2} ) + {{[ T ]}_{0} [ L ]}_{0} {K}_{d 2}^{2} = {0 \\ [ {T}_{2} L ]}_{EQ} = \frac{{{[ T ]}_{0} [ TL ]}_{EQ} - {[ TL ]}_{EQ}^{2}}{{K}_{d 2} + {2 [ TL ]}_{EQ}}$$

in which [T]₀ represents the initial concentration of the target protein; [L]₀ represents the initial concentration of the ligand; [TL]_{EQ} represents the concentration of a monovalently bound target-ligand complex TL at equilibrium, in which the molar ratio of the target protein to the ligand is 1:1; [T₂L]_{EQ} represents the concentration of a divalently bound target-ligand complex T₂L at equilibrium, in which the molar ratio of the target protein to the ligand is 2:1; Kai represents the dissociation constant in the binding of the ligand to the target protein to form the monovalently bound target-ligand complex TL; and K_{d2} represents the dissociation constant in the binding of the monovalently bound target-ligand complex TL to the target protein to form the divalently bound target-ligand complex T₂L (FIG. 7). Other divalent models known in the art may also be used in the present method.

II. High-Throughput Binding Kinetics Measurement

In a second aspect, the present technology provides a high-throughput method for determining binding affinity, the method comprising: (1) preparing a pool of candidate ligands, (2) mixing the pool of candidate ligands with a target protein immobilized on a carrier; (3) isolating the mixture of step (2); (4) sequencing the candidate ligands bound to the target protein to identify a pool of nucleic acid sequences; (5) translating each of the nucleic acid sequences in the pool of sequences identified in step (4); and (6) calculating a frequency of each translated sequence generated in step (5).

In some embodiments, the candidate ligand may be a fusion ligand, an mRNA, a DNA, or a nucleic acid aptamer. The fusion ligand may be a fusion molecule in which a nucleic acid is fused to a protein, a peptide, or a small molecule. In one embodiment, the fusion ligand may be any molecular entity that includes a nucleic acid fused to a protein or a peptide. The nucleic acid may be an aptamer, a DNA, and/or RNA, for example. The RNA may be any RNA, such as mRNA. The protein may be any peptide or protein. In one embodiment, the protein or peptide or small molecule part of the fusion ligand binds to the target protein. The corresponding nucleic acid part of the fusion ligand may then be sequenced. In a particular embodiment, the fusion ligand may be an mRNA-peptide fusion molecule. The methods of preparing the nucleic acid-protein fusion ligands are known in the art. For example, the mRNA-peptide fusion molecules may be prepared according to the methods described in Liu et al., Methods Enzymol. 318, 268-293 (2000) and Takahashi et al., Methods Mol. Biol. 535, 293-314 (2009), the content of all of which are incorporated herein by reference in their entirety. In some embodiments, a pool of mRNA-peptide fusion ligands can be prepared from DNA pools through PCR amplification, in vitro transcription, ligation, and in vitro translation as exemplified in Example 1.

In another embodiment, the candidate ligand may be an mRNA molecule, a DNA molecule, or a nucleic acid aptamer, and the present method may be used to determine the K_{d} of interaction between the mRNA sequence or DNA sequence or the nucleic acid aptamer with its target protein. Technologies that may be useful for selecting the interactions of interest between the target protein and the candidate ligands include, but are not limited to, mRNA display, phage display, ribosome display, yeast display, and aptamer selection.

The carrier can be any suitable substrate on which a protein molecule can be immobilized. As a non-limiting example, the carrier is an ELISA plate and the target protein can be immobilized by a capture-ligand bound to the ELISA plate. In one embodiment, the target protein is a Bcl-xL, which is immobilized to an ELISA plate by a capture-ligand.

Any sequencing technology can be employed by the present method. The sequencing process may include, but is not limited to, next-generation sequencing. Suitable next-generation sequencing technologies include, but are not limited to single-molecule real-time sequencing (Pacific Bio), ion semiconductor (Ion Torrent sequencing), pyrosequencing (454 Life Sciences), sequencing by synthesis (Illumina), sequencing by ligation (SOLiD sequencing), and chain termination (Sanger sequencing). As an example, the next-generation sequencing can be carried out by using HiSeq 2500 System (Illumina). Sequencing results identify all the ligands bound to the beads at that point, allowing the calculation of each candidate ligand's frequency and thus fractional composition. As a non-limiting example, the frequency of any candidate ligand in pool of candidate ligands may be calculated in a process that includes PCR amplification of nucleic acids of the pool of candidate ligands, high-throughput sequencing of the resulting nucleic acids, and subsequent translation of the nucleic acid sequences. The fractional composition may refer to the frequency of the sequence of a particular candidate ligand divided by the total number of sequences in the pool of candidate ligands. Other method of determining the frequency and fractional composition of candidate ligands may also be used in the present method. In one embodiment, the next-generation sequencing process and calculation of the frequency and fractional composition of candidate ligands may be carried out as exemplified in Example 1.

The amount of total candidate ligands bound to the target protein can be calculated by any suitable method. In some embodiments, the amount of candidate ligands are determined by calculating the total amount of ligands bound to the beads as a function of time. Suitable technologies include, but are not limited to, radiolabeling, PCR quantitation, and other methods of quantitation.

In some embodiments, after the frequency of candidate ligands are calculated, the on- and off-rates for the fusion ligands may be calculated. In one embodiment, the fractional composition for each sequence are multiplied by the total amount of ligands bound to the beads as a function of time, which provides the amount of each sequence bound as a function of time. These values can then be fit to the kinetic binding model to achieve the on- and off-rates and ultimately the dissociation constant for each sequence. Any on- and off-rate equations known in the field can be used for the present method, and are within the scope of the present method. As a non-limiting example, the on- and off-rates may be determined by fitting the fractional composition data at various time points (obtained, for example, by radiolabeling) to the formulas below as exemplified in Example 1.

Kinetic on-rate: [C]=[L]₀(1−e^{-k}^{ₒₙ}^{×[T]}^{₀}^{×t})

Kinetic off-rate: [C]=[C]₀e^{-k}^{_{off}}^{×t}

In some embodiments, the present method combines high-throughput DNA sequencing with mRNA display to obtain kinetic on-rates and off-rates, and thus K_{d} values, for tens of thousands of ligands simultaneously.

### EXAMPLES

### Example 1. Methods and Materials

Protein Expression and Purification. The gene for the first 209 amino acids of Bcl-xL (Clone HsCD00004711; Dana Farber/Harvard Cancer Center DNA Resource Core) was PCR amplified with Pfusion polymerase. An N-terminal avitag (AGGLNDIFEAQKIEWHEGG) was added via the PCR reaction for in vivo biotinylation using the BirA enzyme. The product was purified via PCR purification column and cloned into the pET24a vector using NdeI and XhoI. Bcl-xL was expressed overnight at 37° C. in BL21(DE3) cells using auto-induction media. Cells were lysed using Bper (Pierce), and purified using Ni-NTA superflow resin on an FPLC (Bio-Rad), using a gradient from 10 mM to 400 mM imidazole (Buffer A: 25 mM Hepes pH 7.5, 1 M NaCl, 10 mM imidazole; Buffer B: 25 mM Hepes pH 7.5, 1 M NaCl, 400 mM imidazole). Fractions with pure Bcl-xL were combined, concentrated, and desalted into 50 mM Tris-HCl, pH 8.0. Bcl-xL was biotinylated in vitro using BirA biotin ligase (0.1 mg/mL in 50 mM Tris-HCl, pH 8.3, 10 mM ATP, 10 mM Mg(OAc)₂, 50 μM biotin) at 30° C. for two hours. The protein was buffer exchanged into 1×PBS, frozen in liquid nitrogen, and stored at −80° C.

Peptide Synthesis. Peptides E1 (NH₂-MIETITIYNYKKAADHFSMSMGSK-NH₂), E2 (NH₂-MIETITIYKYKKAADHFSMSMGSK-NH₂), D1 (NH₂-MIAISTIYNYKKAADHYAMTKGSK-NH₂), Bim (NH₂-MDMRPEIWIAQELRRIGDEFNAYYARRGK-NH₂), and D79 (NH₂-MIDTNVILNYKKAADHFSITMGSK-NH₂) were synthesized by solid phase Fmoc synthesis, using a Biotage Alstra Microwave Synthesizer. The peptides were synthesized on Rink amide MBHA resin using five-fold molar excess of each amino acid and HATU. After the coupling of the first amino acid, (Fmoc-Lys(Mtt)-OH), the primary amine in the side-chain of the lysine for each peptide was deprotected using a solution of 1% (v/v) trifluoroacetic acid (TFA) in dichloromethane (DCM). Biotin was then coupled to the side-chain primary amine before the synthesis was resumed, resulting in biotin-labeled peptides. Peptides were cleaved from the resin and deprotected with a solution of 95% (v/v) TFA, 2.5% 1,2-ethanedithiol (EDT), 1.5% (v/v) deionized water (DI), and 1% (v/v) triisopropylsilane (TIS) for 2 hours at room temperature. The resin was filtered out, and the peptide was precipitated using 4-fold (v/v) excess ether. The peptides were dried, re-suspended in DMSO, and HPLC purified using a C₁₈ reverse phase column and a gradient of 10-90% acetonitrile/0.1% TFA in water. Fractions were collected and tested for the correct molecular weight using MALDI-TOF mass spectrometry. The correct fractions were lyophilized, dissolved in DMSO, and flash frozen at −80° C.

Radiolabeled Off-Rate Assay. The DNA sequences coding for the peptides were ordered from Integrated DNA Technologies (IDT). Each DNA construct contained a T7 RNA Polymerase promoter, and a 5′ deletion mutant of the Tobacco Mosaic Virus (ΔTMV). The C-terminal portion of the peptides were elongated with a flexible serine-glycine linker (six amino acids long) and an HA tag. After gel purification using urea-PAGE, the DNA sequences were PCR amplified using Taq polymerase and in vitro transcribed into mRNA using T7 RNA polymerase. After transcription, the mRNA was urea-PAGE purified and resuspended in deionized water to a final concentration of 30 μM.

The samples were in vitro translated at 30° C. for 1 hour in the translation solution—150 mM KOAc, 750 μM MgCl₂, 2 μM mRNA, 1× translation mix (20 mM Hepes-KOH pH 7.6, 100 mM creatine phosphate, 2 mM DTT, and 312.5 μM of each amino acid excluding methionine), ³⁵S-labeled methionine (Perkin Elmer; 20 μCi for each 25 μL of translation), and 60% (v/v) rabbit reticulocyte lysate. Radiolabeled peptides were purified using magnetic HA beads (Life Technologies) and eluted with 100 μL, 50 mM NaOH, then immediately neutralized with 20 μL of 1 M Tris-HCl, pH 8.0.

The radiolabeled peptides were allowed to bind to 30 pmol immobilized Bcl-xL for 1 hour in sample buffer (1×PBS, 1% (w/v) BSA, 0.1% (v/v) Tween 20, 10 μM biotin). The beads were magnetically separated, and washed 5× with sample buffer. The beads were resuspended in 1 mL of sample buffer containing 3 μM non-biotinylated Bcl-xL (˜100× molar excess relative to immobilized biotinylated Bcl-xL). At various time points, 100 μL of slurry was removed and the beads were magnetically separated and washed. The percent remaining at each time point was determined by dividing the counts per minute (cpm) on beads by total cpm (beads+washes). The peptide off-rate was determined by an exponential fit of the Percent counts on beads vs. Time (s).

Bead Loading. 54H6 mAb was immobilized on magnetic beads by incubating 400 pmol of the antibody with 1.5 mg of tosyl magnetic beads (Life Technologies) in 1×PBS buffer at 4° C. After 48 hours, the reaction was quenched with 100 μL of 1 M Tris-HCl, pH 8.0. The beads were then washed and re-suspended in 1 mL of 1×PBS+1% (w/v) BSA+0.1% (v/v) Tween-20. Bcl-xL and D1 peptide were immobilized on magnetic beads by incubating 60 pmol of each biotinylated compound with 0.5 mg of streptavidin magnetic beads (Life Technologies) at 4° C. overnight. To block any unbound sites on the streptavidin, 100 nmol of biotin was added and incubated with the beads for 30 minutes at room temperature. The beads were then washed with sample buffer, and resuspended in 600 μL of the same buffer without biotin.

Fluorescein Labeling of the Anti-HIS and Anti-Rabbit Antibodies. Anti-HIS (Thermo Scientific) or Anti-Rabbit (Thermo Scientific) antibodies were buffer exchanged to 1×PBS using a NAP-25 column (GE Healthcare) to remove sodium azide or other preservatives in the storage solution. A twenty-fold molar excess of NHS-fluorescein (Pierce) in DMF was then added to each buffer-exchanged antibody and incubated for one hour at room temperature in the dark. The reactions were quenched with 1 M Tris-HCl, pH 8.0, and buffer exchanged into 1×PBS using NAP-25 columns to remove the unreacted NHS-fluorescein. The concentration of the peptide and anti-HA antibody were calculated as per manufacturer's instructions.

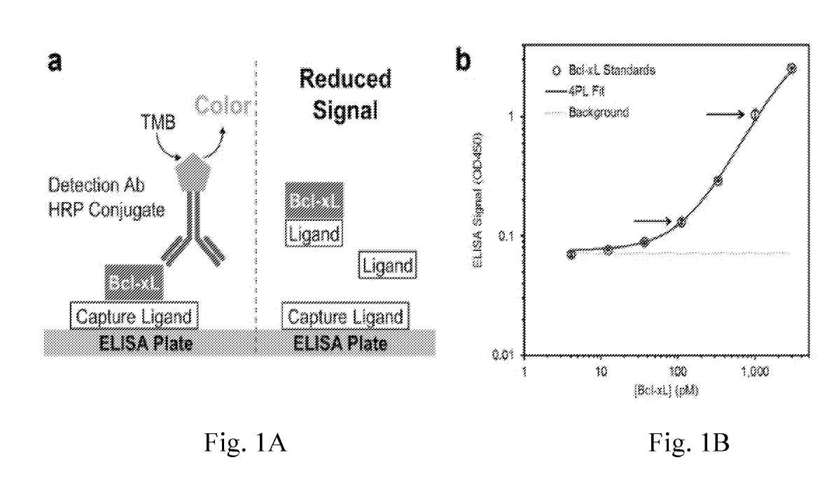

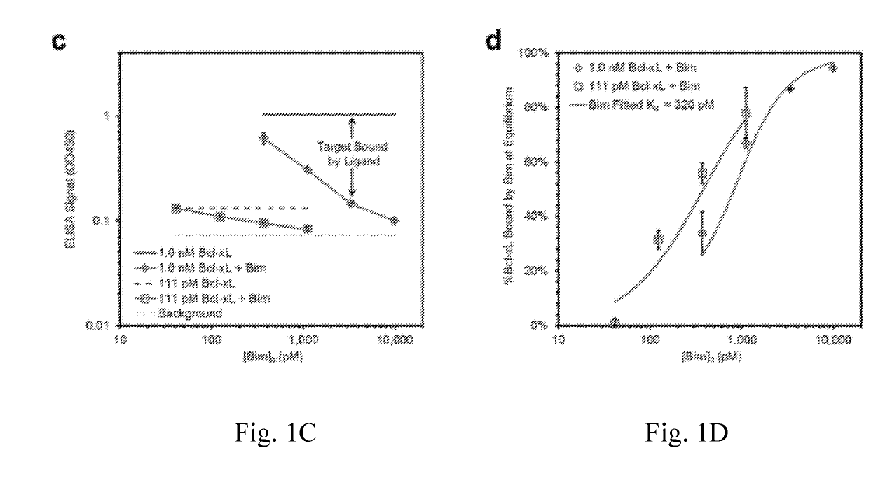

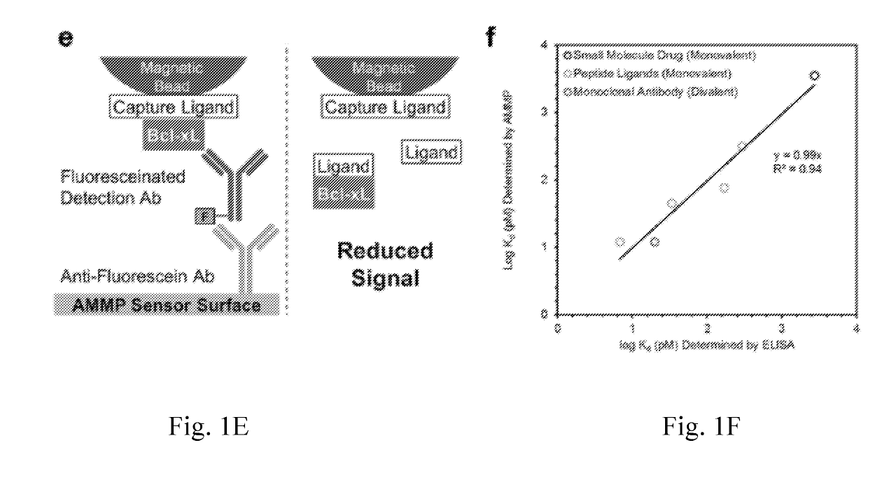

Sample Preparation. A set of serially diluted Bcl-xL standards, at 2× the desired concentration, were made in sample buffer. For each ligand to be tested (such as peptide ligands), a set of dilutions at 2× the desired concentration was also prepared. The Bcl-xL samples were either mixed 1:1 with sample buffer (standards) or ligands (samples), and allowed to incubate at room temperature for 6 days. After the incubation, the standards and samples were analyzed using ELISA or the ViBE BioAnalyzer (FIG. 1).

ELISA Assays. ELISA plates were incubated overnight at 4° C. with 1.5 nmol of streptavidin (for D1 or Bcl-xL capture ligands) or 54H6 mAb in 1×PBS. Plates were washed 3× with wash buffer (1×PBS+0.1% (v/v) Tween-20) and blocked with 1×PBS+5% (w/v) BSA for two hours. For the D1 or Bcl-xL capture ligands, 100 μL of a 30 nM solution of the reagents was added to wells and incubated for 1 hour. This step was skipped for the 54H6 mAb capture ligand (already immobilized on the plate). After the capture ligand incubation, 100 μL of sample or standards were added in each well, and incubated for 1 hour at room temperature. Plates were washed, incubated with HRP-conjugated probe antibody (such as anti-HIS tag antibody) in sample buffer for 1 hour, washed, and incubated with TMB substrate (Thermo Scientific). Reactions were stopped after approximately 10 minutes with 2 M sulfuric acid, and the absorbance at 450 nm was measured via a plate reader (Molecular Devices). The ligand of interest, capture ligand, target protein, and probe ligand used in example ELISA assays performed are highlighted in the table below.

| | | Peptide | mAb (Forward | mAb (Reverse |
|---|---|---|---|---|
| Ligand of interest | ABT 737 | Ligands | Assay) | Assay) |
| Capture Ligand | D1 Peptide | D1 Peptide | 54H6 mAb | Bcl-xL |
| Target | Bcl-xL | Bcl-xL | Bcl-xL | 54H6 mAb |
| Probe Ligand | Anti-HIS-HRP | Anti-HIS-HRP | Anti-HIS-HRP | Anti-Rabbit-HRP |

AMMP Assays. For the AMMP assays, 90 μL of each sample or standards was incubated with 30 μL of magnetic beads (12 μg of beads/mL) and fluorescein-labeled antibody (8 nM) in sample buffer for 1 hour. The experiment's run buffer was 1×PBS+1% (v/v) Tween-20+1% (v/v) heat-treated FBS (Invitrogen; FBS was heat treated for 15 minutes at 65° C. and filtered). BioScale Universal Detection Cartridges were used in performing all of the assays. The device was used per the manufacturer's instructions. The ligand of interest, capture ligand, target protein, and probe ligand used in example AMMP assays performed are highlighted in the table below.

| | | Peptide | Forward mAb | Reverse mAb |
|---|---|---|---|---|
| Assay | ABT 737 | Ligands | Assay | Assay |
| Ligand on Beads | D1 Peptide | D1 Peptide | 54H6 mAb | Bcl-xL |
| Target | Bcl-xL | Bcl-xL | Bcl-xL | 54H6 mAb |
| Probe Ligand | Anti-HIS-Fl | Anti-HIS-Fl | Anti-HIS-Fl | Anti-Rabbit-Fl |

Monovalent and Divalent Analysis. The data for both sets of target concentrations were simultaneously fit for K_{d} (in the K_{d} only fit) or K_{d} and Mo. The data was fitted to the equilibrium model using the lowest absolute deviation method, by varying either only K_{d} or both K_{d} and [L]₀ simultaneously. The monovalent assay fitting was done by Excel Solver (GRG Non-Lin method) using a set of five initial values. The set of values which provided the lowest error after the fitting were chosen as the final values. For the divalent assays, the fitting was performed by MATLAB's fminsearch function and a set of 10 initial values for K_{d1}, K_{d2}, and [L]₀. In order to calculate the % C_{EQ} value, first the concentration of monovalently bound antibody was found by finding the real, positive root of the cubic function in FIG. 7. For the divalent reverse assay, an extra parameter, C_{f}, was also determined by fitting (FIG. 7 Reverse Assay).

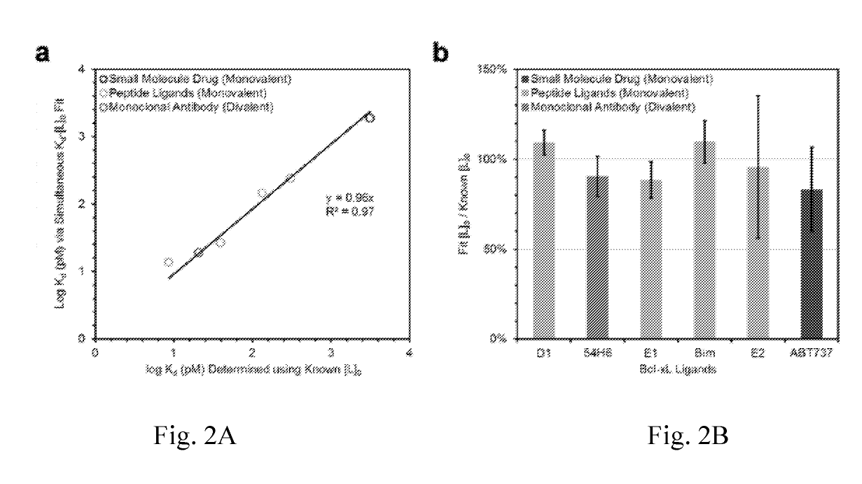

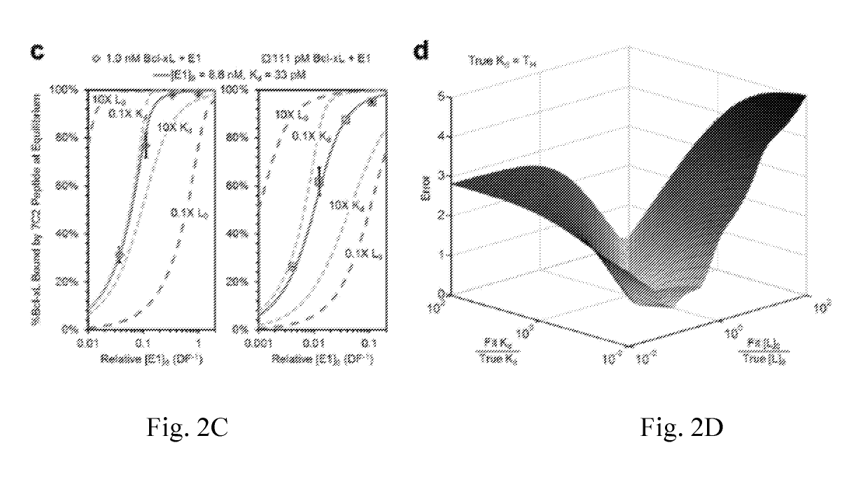

Simulated error analysis. To prepare the 3D-error plot in FIG. 2d, 8 simulated data points were used where two [T]₀ values (T_{H} is high [T]₀ and T_{L} is low [T]₀, and T_{H}=10×T_{L}) and 4 [L]₀ values were chosen (starting from 10×T_{H} diluted serially with a dilution factor of 1:10). A 2D matrix was constructed in MATLAB where the x-coordinate represents the deviation in K_{d} over a 2 order of magnitude window, and the y-coordinate represents the deviation in [L]₀. The total difference (the “error”) between % C_{EQ} when calculated using the deviated K_{d} and [L]₀ values was evaluated against the True K_{d} and [L]₀ values for all 8 data points. The error matrix also depended on the relationship between the true K_{d} value and T_{H}. Six values for K_{d}/T_{H} ratios were tested (100-0.01, going by factors of 10), and the result of one of these (where true K_{d}=T_{H}) is shown in FIG. 2d.

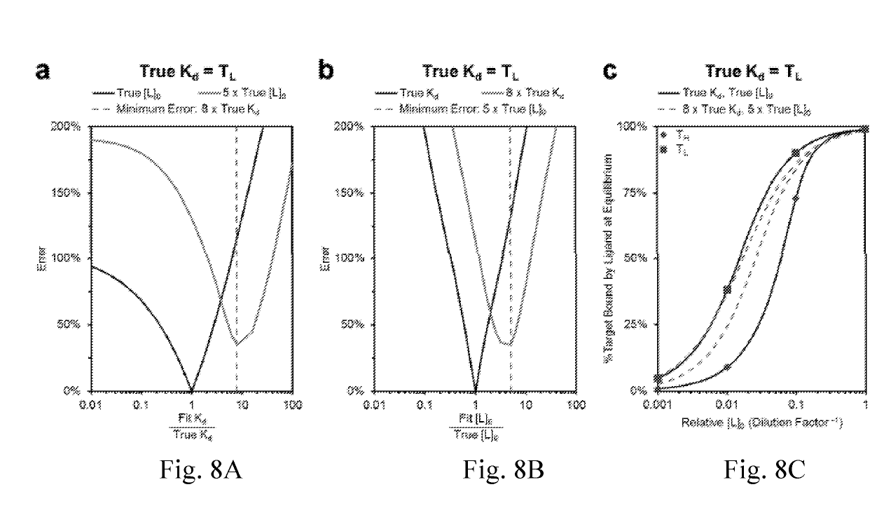

These 2D error matrices were also used in the step-wise analysis for FIG. 8. To perform this type of analysis, a specific column (deviation in K_{d}) was chosen in the matrix. The row with the lowest error for the chosen column represented the optimum [L]₀ value for the specific deviation in K_{d}. If the initial chosen column also represented the lowest error in the optimum [L]₀ row, then the pair of K_{d} and [L]₀ were a stable pair. If not, then the lowest error in the row should be used to find the new optimum deviation in K_{d}, and this iterative method should be continued until a stable pair of values are reached.

Mathematical Formulas. For a monovalent model, FIG. 6 shows formulas governing the equilibrium and transient behavior of a simple binary binding system. The ligand binds to the target to form the target-ligand complex with the rate constant kₒₙ. The complex dissociates back into the target and ligand in solution with the rate constant k_{off}. The total concentration of ligand or target at any point in the reaction is restricted such that the amount in complex ([C]) and the amount free in solution ([L] or [T]) must add up to the initial amount added to the reaction ([L]₀ or [T]₀). The transient solution can be used to ensure enough time has been allocated for the samples to reach equilibrium.

For a divalent model, FIG. 7 shows formulas governing the equilibrium behavior of divalent ligand. The ligand binds to the target to form the monovalently bound target-ligand complex ([TL]) with the rate constant kₒₙ₁. The complex dissociates back into the target and ligand in solution with the rate constant k_{off1}. The monovalently bound target-ligand complex ([TL]) binds to the target to form the divalently bound target-ligand complex ([T₂L]) with the rate constant kₒₙ₂. The complex dissociates back into the target and monovalently bound target-ligand complex ([TL]) with the rate constant k_{off2}. The concentration of the monovalently bound target-ligand complex at equilibrium ([TL]_{EQ}) is the real positive root to the cubic function shown above. The concentration of the divalently bound target-ligand complex at equilibrium ([T₂L]_{EQ}) can be calculated once the [TL]_{EQ} has been found.

Enzymatic K_{d} Calculation Assay. The K_{d} values of ligands of interest were determined using a protocol modified from Friguet et al., Journal of immunological methods, 77, 305-319 (1985). The samples were prepared and analyzed by ELISA assay in a similar process as described above. The OD450 for the standards and their concentration values were fit to a four parameter logistic curve (standard curve). The concentration of the free Bcl-xL in solution (responsible for the signal) for each sample was calculated using the standard curve, and converted into percent of Bcl-xL bound by ligand in solution. For each ligand, the values for all the tested concentration of Bcl-xL and peptide in solution were fit simultaneously to the monovalent equilibrium model below to obtain the dissociation constant K_{d} (in which [C]_{EQ}, [T]₀, [L]₀ represent the concentration of the target-ligand complex at equilibrium, the initial concentration of the target protein, and the initial concentration of the ligand, respectively).

$${[ C ]}_{EQ} = \frac{{[ T ]}_{0} + {[ L ]}_{0} + {K}_{D} - \sqrt{{( {[ T ]}_{0} + {[ L ]}_{0} + {K}_{D} )}^{2} - {{4 [ T ]}_{0} [ L ]}_{0}}}{2}$$

Preparing the pools of fusion ligands. The DNAs for the final enriched pools from the extension and the doped selection against Bcl-x_{L} were generated. The DNA pools were PCR amplified using Taq polymerase and in vitro transcribed into mRNA using T7 RNA polymerase (Liu et al., Methods Enzymol. 318, 268-293 (2000)). After transcription, the mRNA was urea-PAGE purified and resuspended in deionized water to a final concentration of 30 μM. The mRNA was then ligated to fluorescein-F30P (phosphate-dA₂₁-[dT-fluor]-[C9]₃-dAdCdCP; where [dT-fluor] is fluorescein dT (Glen Research), [C9] is spacer 9 (Glen Research), and P is puromycin (Glen Research); synthesized at the Keck Oligo Facility at Yale) using T4 DNA ligase (Takahashi et al., Methods Mol. Biol. 535, 293-314 (2009)). The ligation was performed using a splint complementary to the 3′ end of the RNA and the 5′ end of the DNA-linker. The ligated mRNA was urea-PAGE purified and resuspended in deionized water to final concentration of 30 μM. The samples were in vitro translated in the translation solution-150 mM KOAc, 750 μM MgCl₂, 2 μM mRNA, in 1× translation mix (20 mM Hepes-KOH pH 7.6, 100 mM creatine phosphate, 2 mM DTT, and 312.5 μM of each amino acid) and 60% (v/v) rabbit reticulocyte lysate. To prepare radiolabeled peptides or proteins, non-labeled methionine was substituted with ³⁵S-labeled methionine (Perkin Elmer; 20 μCi for each 25 μL of translation). The translation reactions were incubated at 30° C. for one hour. To form mRNA-protein fusions, KCl and MgCl₂ were added to the reaction to final concentrations of 250 mM and 30 mM respectively after translation, and the samples were frozen at −20° C.

To purify the fusion molecules, 100 μL of dT cellulose (25% (v/v) slurry, GE Healthcare) in isolation buffer (100 mM Tris-HCl pH 8.0, 1 M NaCl, 0.2% (v/v) Triton X-100) was added and incubated for 1 hour. The beads were washed five times with 700 μL of isolation buffer, and the fusions were eluted with 3×80 μL of 65° C. water and desalted through Centrisep columns (Princeton Separations). The desalted fusions were adjusted to 1×RT buffer (50 mM Tris-HCl pH 8.3, 75 mM KCl, 3 mM MgCl₂, 2.4 mM 3′ primer, 200 mM each dNTP,) and the sample was heated to 65° C. for 5 minutes and cooled on ice to anneal the 3′ primer. After cooling, 33.34 of Superscript II enzyme was added and the reaction incubated at 42° C. for one hour. Superscript II was inactivated by heating to 65° C. for 5 minutes, after which the samples were cooled on ice, and used within the same day.

On- and off-rate experiments. To obtain high-throughput sequencing kinetic (HTSK) on-rates, mRNA-peptide fusions of each pool from a 50 μL translation reaction (radiolabeled and non-labeled fusions separately) were first mixed with 7.5 pmols of Bcl-xL immobilized on magnetic beads, and adjusted to 1 mL in 1× Selection buffer (1×PBS, 0.1% (w/v) BSA, 0.1% (v/v) Tween20, 100 μg/mL yeast tRNA, 0.05% (w/v) sodium azide, 10 μM biotin). At each time point, 100 μL of the solution was removed. The non-radiolabeled samples were magnetically separated and washed, PCR amplified with the appropriate primers, and sent for next-generation sequencing. The radiolabeled samples were washed 3×, and the beads were counted via a scintillation counter.

To obtain the HTSK off-rates, after the kinetic on-rate experiment, the remaining beads were washed 5× with selection buffer. The beads were then resuspended in 800 μL of selection buffer without biotin and supplemented with 2 μM Bcl-xL in solution. The excess Bcl-xL in solution prevents binding of dissociated ligands back to the beads. At specific time points, 100 μL of the solution was removed. The non-radiolabeled samples were washed, PCR amplified, and sent for next-generation sequencing. The radiolabeled samples were washed and counted via a scintillation counter.

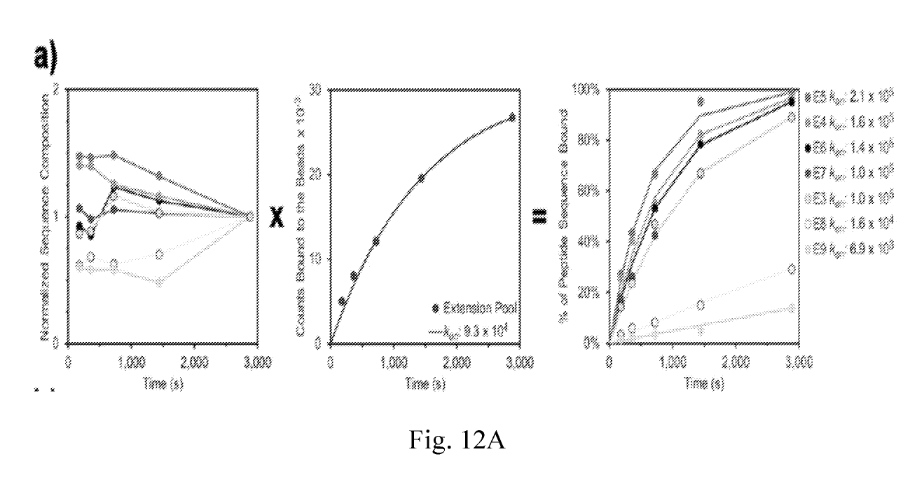

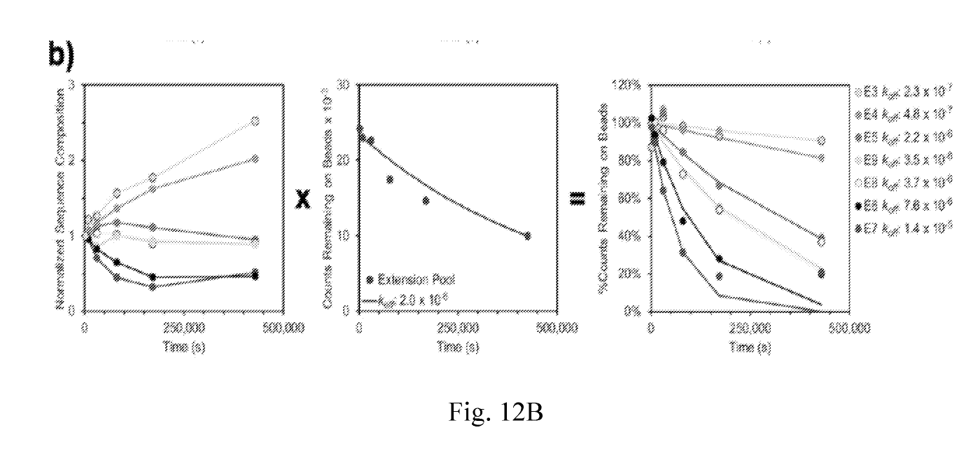

Next Generation DNA Sequencing Analysis. The mRNA-peptide fusions from all of the time points and pools were PCR amplified using unique identifying barcodes, combined into a single sample and sent for high-throughput DNA sequencing using a HiSeq 2500 machine at the USC genome core. The file containing the results from the DNA sequencing run (FASTQ format) was first stripped of all content except for the DNA sequences using python code developed in house. Then the file was split into separate files for each on- and off-rate time point based on the DNA bar code. Each DNA sequence in each file was then translated (only the region after the start codon until the 3′ primer, using biopython and in house developed code) and the frequency of each translated sequence in the pool was calculated. Then, the fractional composition (frequency of the sequence divided by the total sequences in the pool) for each sequence was calculated. A separate file was created per selection to track the frequency composition for each sequence throughout the various time points. An example of this data can be seen in FIGS. 12a and 12b in the left panels.

Obtaining the on- and off-rates by HTSK. To obtain the on-rate for each sequence, the fractional composition for each sequence was multiplied by the radiolabeled counts for that pool's time point. This results in the radiolabeled counts per sequence as a function of time. These values (representing [C]), the concentration of immobilized Bcl-xL on magnetic beads, and time in seconds were fit to the on-rate equation shown below to obtain [L]₀ (asymptotic maximum) and kₒₙ for each sequence. The fitting was done using the fminsearch function in MATLAB to minimize the error (Least Absolute Deviation method) between the real data and the model by changing [L]₀ and kₒₙ. To obtain the off-rate, the same procedure was performed with the off-rate portion of the fraction composition data for each sequence. MATLAB was used to fit the product of the fractional composition and the radiolabeled pool counts at each time point, to the off-rate formula shown below.

Kinetic on-rate: [C]=[L](1−e^{-k}^{ₒₙ}^{×[T]}^{₀}^{×t})

Kinetic off-rate: [C]=[C]e^{-k}^{_{off}}^{×t}

Due to the relatively short time period for the on-rate segment of the experiment (˜45 minutes) and the very slow off-rate for the clones (˜2×10⁻⁶ on average) the contribution from the off-rate can be ignored during the binding phase. This allowed the transient complex concentration equation under excess target concentration conditions to reduce to the kinetic on-rate expression above. To fit the HTSK data to the above model, the % C bound as a function of equilibrium value was obtained by dividing [C] by [L]₀. This allowed the fitting of the data for 2 parameters: % Cₘₐₓ and kₒₙ. The kinetic off-rate was obtained by blocking the on-rate contribution to the transient binding model. The k_{off} value was obtained by fitting the HTSK data for 2 parameters: % Cₘₐₓ and k_{off}.

To obtain the on- and off-rates for each sequence without using the radiolabeled data, it is possible to use another method of quantitating the amount of pool bound to the beads at each time point. The amount of DNA bound to the beads was quantified by measuring the intensity of the DNA bands in the agarose gels using ImageJ's intensity measurement tool, and using the DNA ladder (NEB 100 bp ladder) as our standards.

The number of sequences that this analysis can give reliable results for depends on diversity and the status of the library. Only the ligands with a statistically significant representation in a pool were analyzed. For a pool that had converged to a large degree (extension pool), where the top 50 sequences accounted for ˜78% of the pool, HTSK results were obtained for approximately 2,000 sequences. However for a less converged pool (Doped) where the top 50 sequences accounted for ˜3% of the pool, HTSK results were obtained for 20,000 sequences. The HTSK analysis could not, however, provide kinetics constants for any sequences if the diversity of the pool was too high (where the highest represented sequence in the library accounted for less than 1 PPM of the library).

### Example 2. Simultaneous Determination of K_{d} and [L]₀

Bcl-xL was used as the target protein. This protein has three distinct classes of known ligands—antibodies, peptides, and small molecules. Ligands used were a commercial monoclonal antibodies (54H6), a small molecule compound (ABT-737), and a synthetic 26-residue fragment of Bim (a pro-apoptotic natural ligand of Bcl-xL12), and three ultrahigh affinity peptides (K_{d}<1 nM) that bind to Bcl-xL. In some embodiments, the peptides and small molecule compounds bind one site in Bcl-xL and the antibody binds a second, noncompeting site on the protein.

Forward (Target in Solution) Equilibrium Assay

A forward equilibrium assay was conducted to determine the K_{d} for the ligands listed above (54H6, ABT-737, and Bim). The equilibrium assay is a modified version of the method described by Friguet et al., supra. The samples were prepared and analyzed by ELISA assay in a similar process as described above. As shown in FIG. 1a, a capture ligand was used to pull down the free target protein in solution. A competing ligand (Bim in FIG. 1) of unknown K_{d} was incubated with the target and allowed to equilibrate. As shown in FIG. 1a, target protein bound to the competing ligand is not anchored to the ELISA plate. Subsequent wash steps thereby reduce the amount of free target in solution. The K_{d} of interaction between the target protein and the competing ligand can then be determined by quantifying the amount of free target in solution. Based on the response curve for target quantitation (shown in FIG. 1b), two target concentrations were chosen that gave signal that was above background yet not saturated (111 pM and 1 nM, indicated with arrows) for further analysis. At each of these concentrations, the competing ligand was equilibrated with the sample to reduce the signal (FIG. 1c). These data were fit to yield a single K_{d} and result in two curves that corresponded to the different target concentrations (FIG. 1d). The equilibrium models for monovalent and divalent ligands are shown in FIGS. 6 and 7.

The forward assay was also carried out by AAMP in a similar process as described above using a commercially available quantitation platform, the ViBE BioAnalyzer, capable of high-throughput automatic sample analysis (FIG. 1e). As shown in FIG. 1e, the capture ligand was used to pull down the free target protein in solution. A competing ligand of unknown K_{d} was incubated with the target and allowed to equilibrate. The target protein bound to the competing ligand is not anchored to the magnetic beads. Subsequent wash steps thereby reduce the amount of free target in solution. The K_{d} of interaction between the target protein and the competing ligand can then be determined by quantifying the amount of free target in solution. Comparing the AMMP (ViBE Platform) and ELISA methods demonstrated that antibody, small molecule, and peptide ligands gave the same K_{d} values independent of the measurement method (Figure if and Table 2). These results validate the AMMP approach for K_{d} measurements as the accuracy of the equilibrium ELISA method has been shown extensively. Additionally, the K_{d} value for the Bim peptide (130±40 pM) measured in the present method matches the reported value in the literature (140 pM) (Sleebs et al., J Med Chem, 56, 5514-5540 (2013)), and the calculated kₒₙ values for all tested peptides fell within 10⁴-10⁶ (M⁻¹s⁻¹) typically observed for most protein-protein interactions (Table 3).

Measuring K_{d} where the Ligand Concentration is Unknown

The K_{d} values for Bcl-xL ligands and the value of [L]₀ for each of the ligands were determined (FIG. 1). The same data were re-analyzed without inserting the value of [L]₀, to determine both K_{d} and [L]₀ simultaneously. Remarkably, the results showed the same values of K_{d} (FIG. 2a) and [L]₀ (FIG. 2b) as those obtained using standard approaches for all three classes of ligands. The correspondence between the two approaches was excellent, giving the same values of K_{d} over the entire range studied.

Fidelity of the Fit and Parameter Sensitivity

The sensitivity of the fitting process to each of the input values of K_{d} and [L]₀ was examined. FIG. 2c shows a rudimentary measure of the fidelity of each parameter. After obtaining K_{d} and [L]₀ values through simultaneous fitting, one parameter was kept constant and the other parameter was changed by an order of magnitude in each direction to show the accuracy of the obtained values (light and dark gray dashed lines).

FIG. 2c indicates that the fit values for K_{d} and [L]₀ are correct. When a pair of K_{d} and [L]₀ values are fit, the error between the data and the equilibrium model was plotted as one parameter is fixed, and the other was scanned over a range. Values were accepted when each parameter produces the minimum level of error when the other parameter is fixed (FIGS. 8a and 8b). However, this iterative fitting analysis cannot show how changing one parameter can compensate for changing the other. This approach can result in self-consistent pairs of K_{d} and [L]₀ that are incorrect and far from true K_{d} and [L]₀ values (such as the example shown FIG. 8c).

Fitting for two variables simultaneously can result in a situation where varying one parameter can compensate for the error generated when the other parameter is moved. To address this problem, a more rigorous analysis of parameter sensitivity was carried out. The overall error changes for all combinations of fit K_{d} and [L]₀ values were examined. Given the true K_{d} and [L]₀, and two target concentrations each with 4 dilutions of ligand, 8 data points were simulated. The K_{d} and [L]₀ were then varied within a four orders of magnitude window, and binding percentages was calculated at equilibrium. Error was defined as the total distance between the two sets of data points (FIG. 2d). This type of analysis produced an error surface where the z-axis corresponds to the error and the x- and y-axis values show the changes in K_{d} and [L]₀ using the true values of each as a reference point. Hence, at the center of the plot (where K_{d} and [L]₀=their true values) the error (z-axis) was defined as zero.

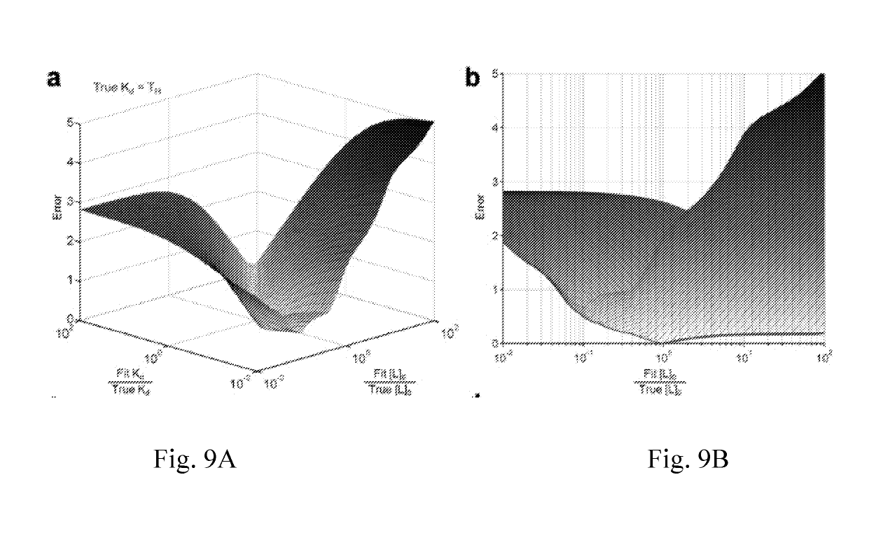

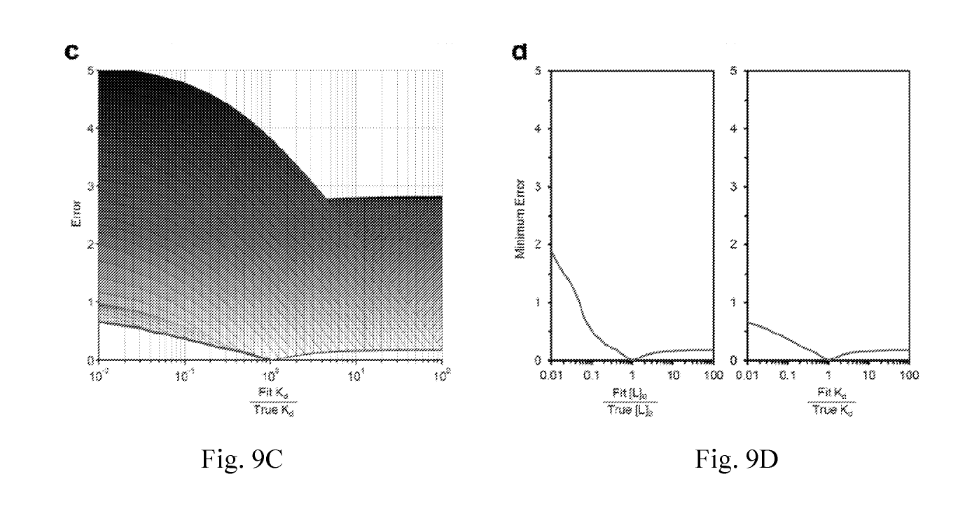

As shown in FIG. 2d, many different combinations of [L]₀ and K_{d} resulted in relatively large error values. The error surface approached the x-y plane (where error is lowest) for a very restricted set of values of both parameters—the ravine running down the middle of the surface. This approach to viewing the data obscures whether there is a unique solution where error is minimized, or whether there are a family of solutions of K_{d} and [L]₀ that give error values very near the x-y plane. To address this, the error surface (FIG. 2d) was projected onto the [L]₀-error plane (FIG. 2e) or the K_{d}-error plane (FIG. 20, and only the lowest error values for each projection was retained (details shown in FIG. 9). A point on each line in FIG. 2e thus represents the minimum error for a given variation in [L]₀, over all tested K_{d} values. The lines corresponding to the error surface in FIG. 2d are shown in FIGS. 2e and 2f (purple dashed lines).

Thus, the accuracy of this analysis depends on the K_{d} value in relation to the concentrations of the target (low target concentration—T_{L} and high target concentration—T_{H}) used in the experiments. The results in FIGS. 2e and 2f produce a unique, unambiguous solution approaching the x-axis at a single point, the true value of [L]₀ and K_{d} respectively. Some choices of target concentrations vs. K_{d} were analyzed to provide clear solutions (results shown in FIG. 2e (blue, green, and orange curves) and FIG. 2f (orange curves)). Some choices of target concentrations gave ambiguous results, and cannot be used to determine accurate values of K_{d} and [L]₀ (FIGS. 2e and 2f, red curves).

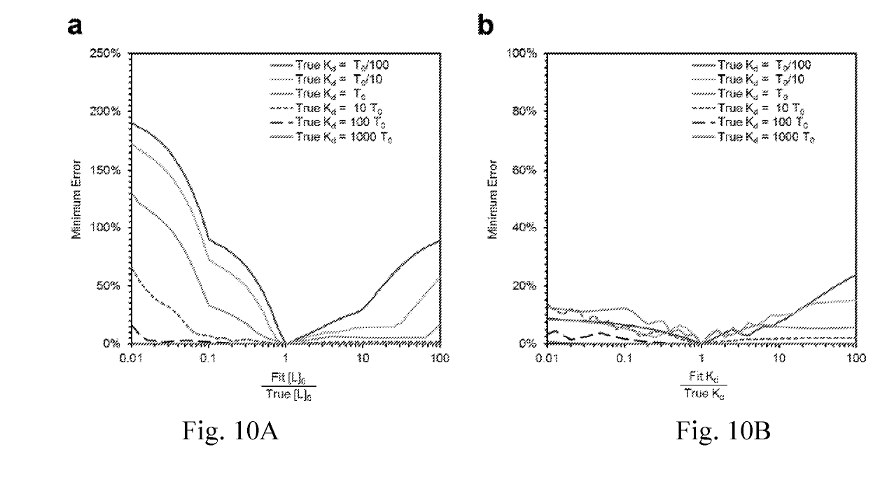

This type of analysis can be formulated as a set of rules that direct where K_{d} and [L]₀ can be determined. When the high and low target concentrations are 10-fold apart and the ligand concentration ranges from 10×T_{H} to 0.1×T_{L}, accurate K_{d} values can be obtained for T_{H}>K_{d}>0.1×T_{L}. The accuracy of fit [L]₀ follows a significantly different rule: the fit for [L]₀ is accurate when T_{H}>K_{d}, and is improved continuously as the K_{d} is lowered with respect to initial target concentration. These ranges are guidelines for assessing the accuracy of the obtained K_{d} and [L]₀ values. If the obtained K_{d} value is within the T_{H}>K_{d}>0.1×T_{L} range, the fits can be trusted. However, if the obtained K_{d} is outside the window, the experiment must be repeated with new initial target concentrations. This same type of analysis can be used to demonstrate that accurate K_{d} and [L]₀ values cannot be determined using a single target concentration (FIG. 10), showing that at least two concentrations of target are needed.

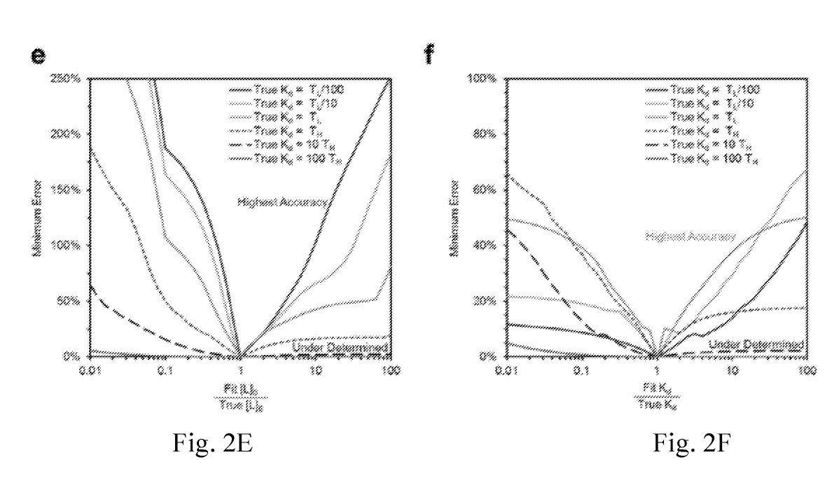

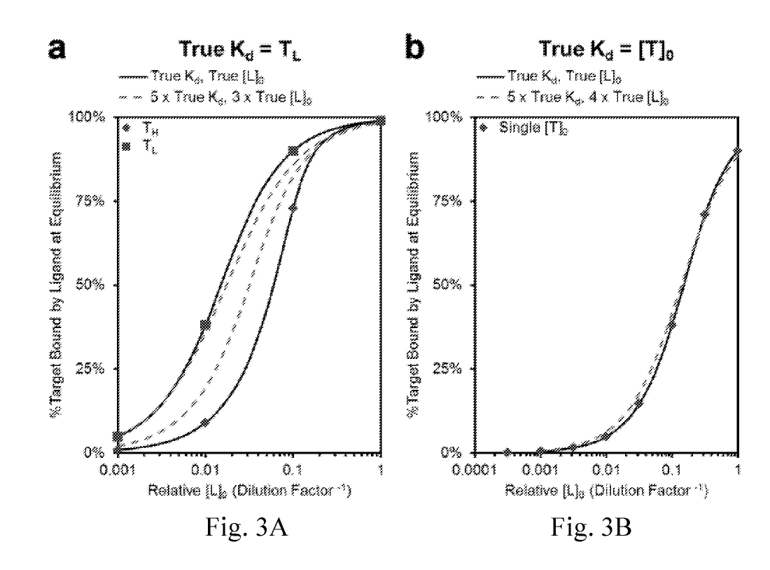

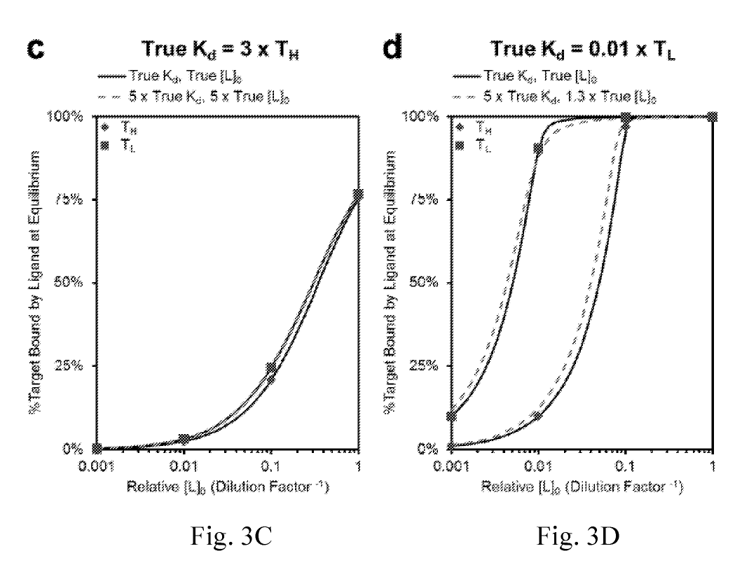

The validity of the above ranges is shown in FIG. 3. When the true K_{d} is within the optimum range, a 5-fold deviation in fit K_{d} cannot be compensated for by adjusting the [L]₀ value (FIG. 3a). Here, the erroneous K_{d} and [L]₀ values do not fit the data. However if a single target concentration is used (FIG. 3b), or K_{d} is outside the specified range (FIGS. 3c and 3d), the data points and the erroneous K_{d} and [L]₀ values match and would be falsely interpreted as “correct” K_{d} and [L]₀ values.

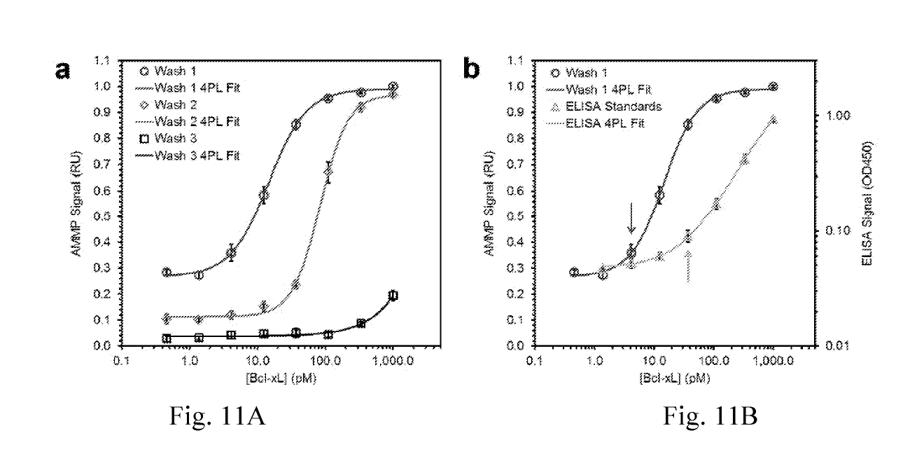

Any experimental method that extends the quantitative range of the response curve (for example, vs. standard ELISA) provides a means to determine high affinity binding constants with high accuracy. Commercial AMMP device was used for some of the analysis to provide this extended range. The AMMP assay is more sensitive than the ELISA (FIG. 11) and on average yielded a ˜5-fold increase in sensitivity. The higher sensitivity of the AMMP assay makes K_{d} measurements possible even with sub-picomolar interactions.

Treating Antibodies as Divalent Ligands

Data were systematically fit in the forward and reverse assays with monovalent and explicit divalent models, toward the goal of quantitating valency effects and developing a useful version of the reverse assay.

Divalent Ligands: Forward Assay

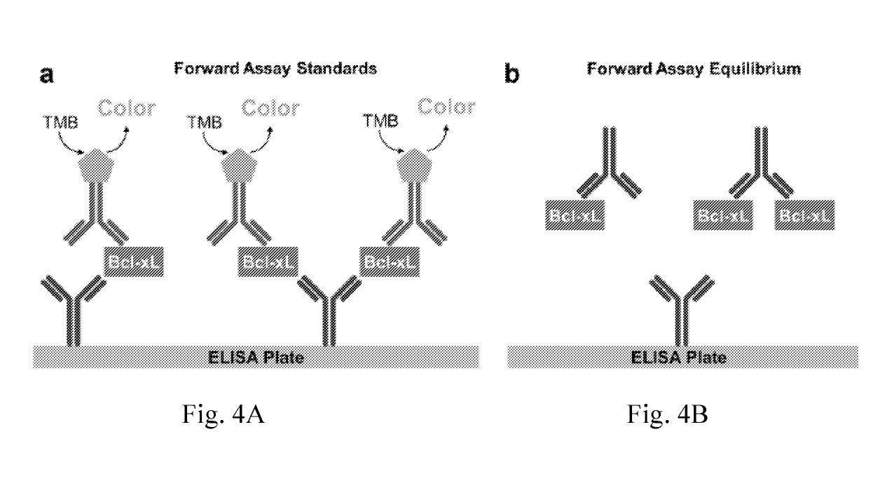

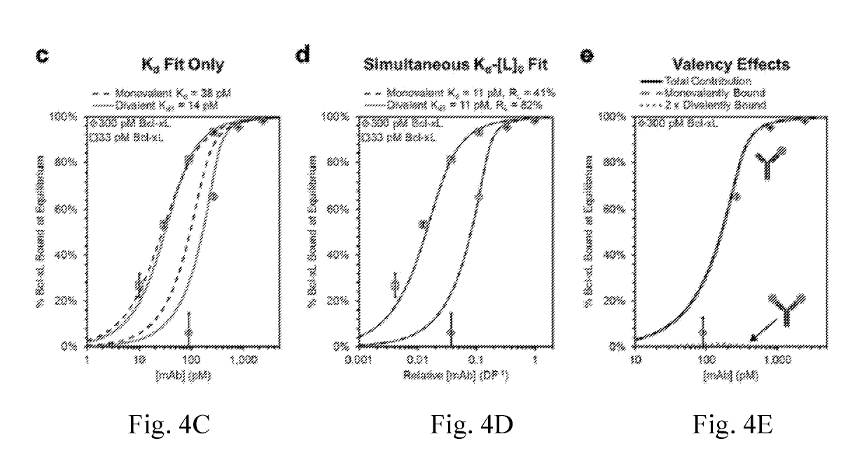

The forward assay (FIGS. 4a and 4b) was conducted in the same manner for both monovalent and divalent ligands. When only fitting for K_{d}, the divalent model provides better fits for the data than the monovalent model (FIG. 4c) and gives markedly different results for K_{d} (38 pM for the monovalent model vs. 14 pM for the divalent model). When fitting for both K_{d} and [L]₀ simultaneously (FIG. 4d), both models give curves that fit the data well and produce K_{d} values identical to the divalent K_{d}-only fit (K_{d}=11 pM). However, the monovalent model produces a fit [L]₀ that is equivalent to the antibody concentration and thus half of the total concentration of sites. These data indicate that for the forward assay to give accurate K_{d} values, one must use the antibody concentration (rather than the number of sites) with the monovalent equilibrium model, a marked change from current practice. This is due to the negligible contribution of the divalently bound ligand at equilibrium for the forward assay (FIG. 4e), essentially turning antibodies into monovalent ligands under these conditions.

As shown above, a pair of erroneous K_{d} and [L]₀ values can match the data points when a single target concentration is used. Since most equilibrium immunoassays to determine antibody K_{d} values use a single target concentration, previous studies have failed to uncover this discrepancy. This issue is only observed when multiple concentrations of target are used, however it is often simply attributed to ligand activity. An activity coefficient of 0.5 is often obtained, suggesting that half of antibody sites are non-functional (mean activity coefficient for various antibodies reported as 0.47±0.07 and 0.53±0.05) (Bee et al., 2012, supra; Bee et al., 2013, supra).

Divalent Ligands: Reverse Assay

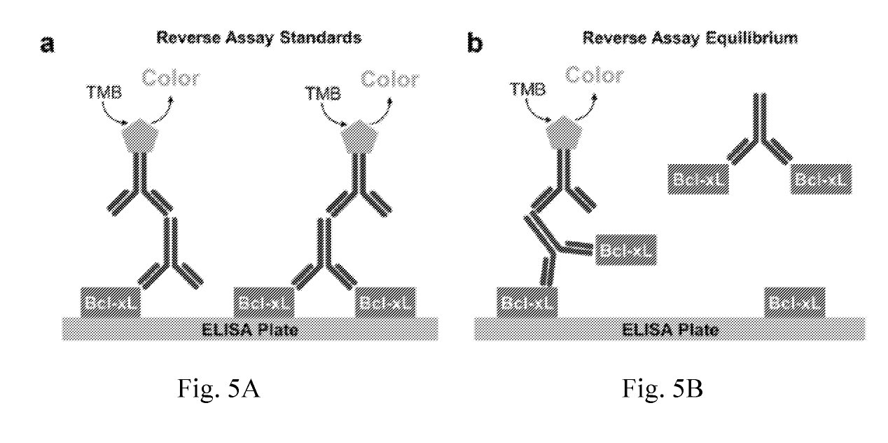

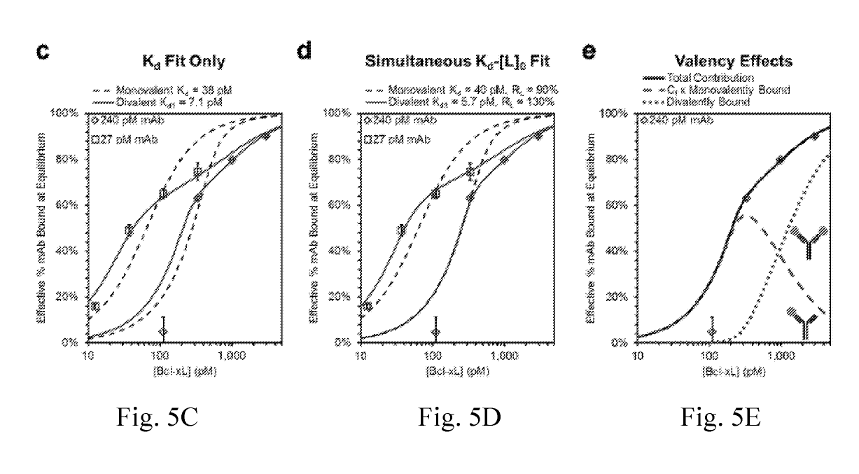

The schematic approach for the reverse assay is shown in FIGS. 5a and 5b.

Unlike the forward assay, in the reverse assay the target is immobilized and used to capture the free ligand in solution (FIG. 5a). The main difference between the forward and the reverse assay is that for multivalent ligands, monovalently bound ligands are still able to interact with the immobilized target (FIG. 5b). The strength of this interaction depends on the cooperativity of the binding sites as well as the immobilized target density. Due to this effect, the use of the reverse assay has been discouraged in the past. For the divalent equilibrium model, the present method adds a cooperativity term to account for the strength of interaction between the target and a free ligand vs. a monovalently bound ligand. The cooperativity factor (C_{f}) measures the percent of the monovalently bound ligand which does not interact with the immobilized target. This means that for the divalent model, the effective complex concentration at equilibrium is the concentration of the divalently bound ligand (unable to interact with the immobilized target) plus the concentration of the monovalently bound ligand multiplied by the cooperativity factor (concentration of the monovalently ligand which is unable to interact with immobilized target).

Similar to the forward assay, two concentrations of the species in solution (here, the monoclonal antibody) were used to obtain accurate K_{d} and [L]₀ values. Data from a sample reverse assay is shown in FIG. 5c. When the high and low ligand concentrations are fit to equilibrium models, only the divalent model simulates the behavior of the obtained data points. Interestingly, simultaneously fitting for both K_{d} and [L]₀ does not help the monovalent model match the data better than fitting for K_{d} only (FIG. 5d). For the reverse assay, both the monovalently bound and divalently bound species are present at significant quantities and contribute to the effective complex composition at equilibrium. While at low target concentrations the monovalently bound ligand dominates the signal, at high target concentration the divalently bound ligand has the most significant contribution (FIG. 5e). The cooperativity constant depends on several factors such K_{d1}, K_{d2}, and immobilized target density. The value of the cooperativity factor was obtained by fitting and remained consistent for all experiments: 74%±4% for the K_{d} fit only and 73%±3% for the simultaneous K_{d}−[L]₀ fit.

While the data from the forward assay is convincing that divalent modeling of the antibody is more accurate than monovalent modeling with twice the concentration, it is still possible that the antibody was simply ˜50% inactive. Obtaining accurate K_{d} and [L]₀ values from the reverse assay that match the forward equilibrium assay solves a persisting problem in the field and removes any doubt that the antibody is not inactive, rather, all antibody sites are functional.

Table 1 shows the measured K_{d} values and [L]₀ ratios for the tested ligands. Mean K_{d} values and [L]₀ ratios with associated standard errors are reported. The data are from both the ELISA and the AMMP assays. For the mAb, K_{d1} refers to the dissociation constant for the free mAb for Bcl-xL. The K_{d1} values for the 54H6 mAb are obtained by combining the data from both forward (target in solution) and reverse (target immobilized) assays. The mAb K_{d2} values were obtained using only the reverse assay, as the divalently bound species was a significant contributor to the overall results in this format.

TABLE 1

| | Equilibrium | K_{d} Determined Using | K_{d} Determined by | Ratio of Fit [L]₀ to |
|---|---|---|---|---|
| Ligand | Model | Known [L]₀ (pM) | Fitting for [L]₀ (pM) | Known [L]₀ |
| D1 Pep | Monovalent | 8.5 ± 2 | 14 ± 5 | 109% ± 7% |
| E1 Pep | Monovalent | 39 ± 6 | 27 ± 12 | 88% ± 10% |
| Bim Pep | Monovalent | 130 ± 40 | 150 ± 80 | 110% ± 12% |
| E2 Pep | Monovalent | 300 ± 14 | 240 ± 94 | 96% ± 40% |
| ABT-737 | Monovalent | 3,100 ± 360 | 1,900 ± 790 | 83% ± 24% |
| 54H6 mAb | Divalent | K_{d1} = 21 ± 6 | K_{d1} = 19 ± 4 | 90% ± 11% |
| | | K_{d2} = 3,300 ± 1,300 | K_{d2} = 4,000 ± 1,800 | |

Table 2 shows the K_{d} values for the ligands as determined by the ELISA or the AMMP assays. Mean values and standard errors are reported.

TABLE 2

| | Equilibrium | K_{d} Determined by | K_{d} Determined by |
|---|---|---|---|
| Ligand | Model | ELISA (pM) | AMMP (pM) |
| D1 | Monovalent | 7 ± 2 | 12 ± 2 |
| E1 | Monovalent | 34 ± 9 | 45 ± 8 |
| Bim | Monovalent | 170 ± 55 | 77 ± 11 |
| E2 | Monovalent | 290 ± 29 | 315 ± 9 |
| ABT-737 | Monovalent | 2,700 ± 260 | 3,500 ± 660 |
| 54H6 | Divalent | 20 ± 5 | 12 ± 7 |

Table 3 shows the calculated kinetic on-rate for Bcl-xL binding peptides. The K_{d} for the peptides is measured by the equilibrium ELISA/AAMP assays (Table 1). The off-rate for these peptides was obtained by measuring the dissociation rate for radiolabeled peptide-mRNA fusions bound to immobilized Bcl-xL. The on-rate was calculated based on the equilibrium K_{d} measurements and the radiolabeled off-rate.

TABLE 3

| Ligand | Type | K_{d} (M) | k_{off} (s⁻¹) | kₒₙ (M s⁻¹) |
|---|---|---|---|---|
| D1 | Peptide Ligand | 8.5E−12 | 2.0E−6 | 2.4E5 |
| E1 | Peptide Ligand | 3.9E−11 | 1.2E−5 | 3.1E5 |
| Bim | Peptide Ligand | 1.3E−10 | 1.2E−4 | 9.2E5 |
| E2 | Peptide Ligand | 3.0E−10 | 1.6E−5 | 5.3E4 |

Thus, both K_{d} and [L]₀ values for a ligand-target interaction were determined simultaneously. The validity of the process was tested by performing detailed error analysis, which demonstrates that the fitting of the present method gives unique and reproducible solutions. Further, the above process defined where K_{d} and [L]₀ measures are reliable and where they are underdetermined. By using a divalent equilibrium model for antibody binding, the above process shows that obtaining reliable K_{d} and [L]₀ values is only possible when the cooperativity factor between the two antibody binding sites has been taken into account.

### Example 3. High-Throughput Binding Kinetics Measurement

Two enriched pools of fusion ligands were chosen against Bcl-xL as a target protein. The enriched pools include an extension selection pool, and a doped selection pool. The extension selection pool contained peptide ligands against Bcl-xL that are 21 amino acids long. The doped selection pool contains top ranking sequences from the extension selection pool, and is used to create a biased library to further optimize binding affinity. The mRNA of both pools were ligated to a 3′ DNA linker attached to puromycin, in vitro translated, purified and reverse transcribed to prepare a library of mRNA-peptide fusions. A small fraction of each pool are also translated using radiolabeled methionine to provide a library of radiolabeled mRNA-peptide fusions that can be used to track the binding of the mRNA-peptide fusions in the pool to the target protein.

To obtain high-throughput sequencing kinetic (HTSK) on-rates, a library of mRNA-peptide fusions were first mixed with Bcl-xL immobilized on magnetic beads. The mixture (containing the magnetic beads with Bcl-xL and the mRNA-peptide fusions bound to the Bcl-xL target) was isolated at a series of predetermined time points for further analysis. A portion of the beads were removed at various time points, washed, PCR amplified, and sent for sequencing by next-generation sequencing using HiSeq 2500 System (Illumina) (FIG. 12a, left panel). After the kinetic on-rate determination experiments, the beads were washed and excess target was added in solution to inhibit re-binding of ligand molecules to the beads before continuing the kinetic studies at further time points.

The ligands bound to the beads were identified by sequencing, allowing the calculation of each ligand's frequency and thus fractional composition. The total amount of ligands bound to the beads as a function of time was determined by radiolabeling. After the frequency of sequences were calculated, in order to calculate the on- and off-rates for the ligands, the fractional composition was multiplied by the amount of ligands bound to the beads as a function of time, which provides the amount of each sequence bound as a function of time. These values were then fit to the kinetic binding model to achieve the on- and off-rates and ultimately the dissociation constant for each sequence.

By separately using the radiolabeled samples, the amount of peptide bound to the beads at each time point are measured (FIG. 12a, middle panel). The amount of radiolabeled binding at each time point represents the sum of all the peptides bound to the beads at that point. To obtain the kinetic on-rates for each ligand, each ligand's fractional composition was multiplied by the total radiolabeled binding. This results in a measure of binding for each sequence as a function of time (FIG. 12a right panel). Based on this analysis and the concentration of the immobilized Bcl-xL, the kinetic on-rate for each sequence was obtained by fitting the binding data to a simple kinetic on-rate equation. The contribution of the dissociation-rate to the binding equation was removed because in the small time scale of this experiment (˜45 minutes) and given the slow off-rate of the sequences tested (2×10-6 s⁻¹ on average), the contribution of the dissociation rate was minimal. This allowed for independent calculation of on- and off-rates. The kinetic on- and off-rates can be calculated based on the equations shown in the Materials and Methods section above.

A similar approach was employed to obtain the HTSK off-rates. After the kinetic on-rate experiment, the remaining beads are washed and excess Bcl-xL was added in solution to prevent binding of dissociated ligands back to the beads. Periodically, a fraction of beads were removed, washed, PCR amplified, and sent for next-generation sequencing (FIG. 12b left panel). Then, each sequence's fractional composition was multiplied by the total radiolabeled peptides still bound at each time point to obtain the amount of each peptide still bound as a function of time (FIG. 12b, middle panel). A simple exponential fit was then used to calculate the kinetic off-rate (FIG. 12b, right panel).

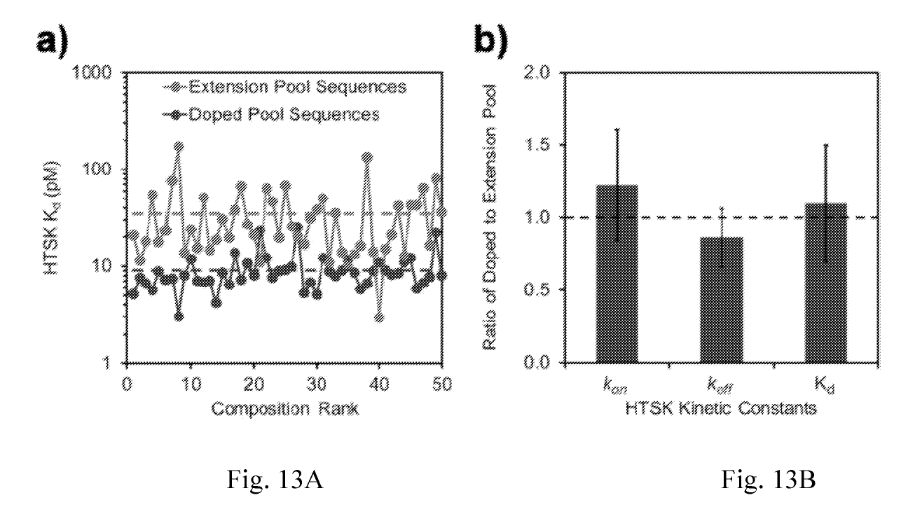

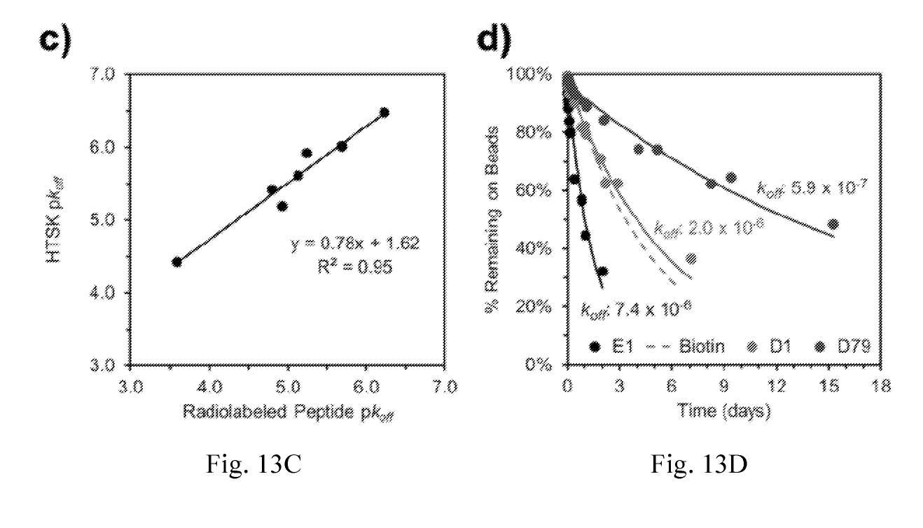

FIG. 13a shows the K_{d} obtained for the 50 highest frequency ligands in each tested pool. The ligands in the doped pool show a higher affinity on average than the ligands in the extension pool. The results show that the frequency rank poorly correlates to sequence affinity. Further, the values for the 40 ligands that appeared in both the extension and doped pools are compared to show the reproducibility of the kinetic constants obtained by the present method (FIG. 13b). The results show that the HTSK values are remarkably reproducible and highly precise.

Furthermore, the off-rates of several ligands were tested using in vitro translated radiolabeled peptides to verify the validity of the results obtained by the present method. The peptide ligands were made using a C-terminal HA tag, and affinity purified. The off-rate of the radiolabeled peptides was then determined using similar methods as the radiolabeled pool off-rate. FIG. 13c shows the HTSK vs. radiolabeled peptide off-rates. The HTSK off-rates correlate very well to the radiolabeled peptide off-rates, however, there is a consistent bias between the two methods. The measured bias is small and is less in comparison to biases measured between other established methods for affinity. One contributing factor to this difference could be the context of binding. The HTSK results are obtained for mRNA-DNA-peptide fusion molecules whereas the radiolabeled k_{off} values are for the peptide with a short C-terminal HA tag. Table 4 shows the validity of the HTSK results. The kinetic off-rates and the dissociation constant for three selected clones were obtained by HTSK, and were compared to the results obtained by radiolabeled peptides (k_{off}) and ELISA (K_{d}).

TABLE 4

| | | Peptide | HTSK | Enzymatic | HTSK |
|---|---|---|---|---|---|
| | Sequence | k_{off} (s⁻¹) | k_{off} (s⁻¹) | K_{d} (pM) | K_{d} (pM) |
| E1 | MIETITIYNYKKAADHFSMSM | 7.4 × 10⁻⁶ | 2.5 × 10⁻⁶ | 39 ± 6* | 23 ± 2 |
| D1 | --AIS-----------YA-TK | 2.0 × 10⁻⁶ | 1.0 × 10⁻⁶ | 9 ± 2* | 15 |
| D79 | --D-NV-L----------IT- | 5.9 × 10⁻⁷ | 3.3 × 10⁻⁷ | | 2.4 |

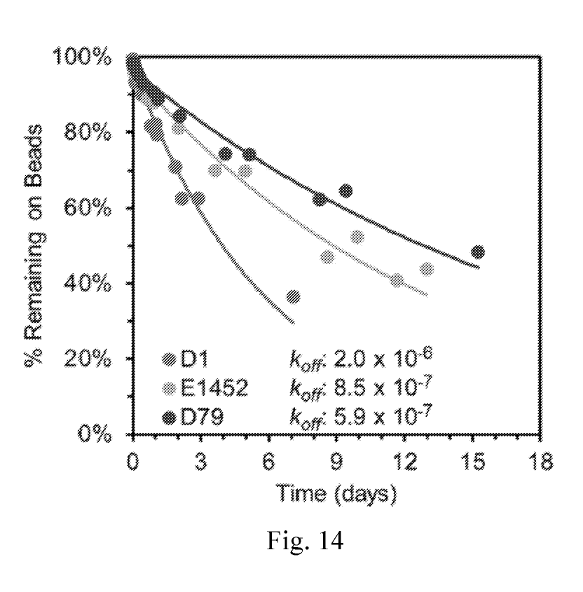

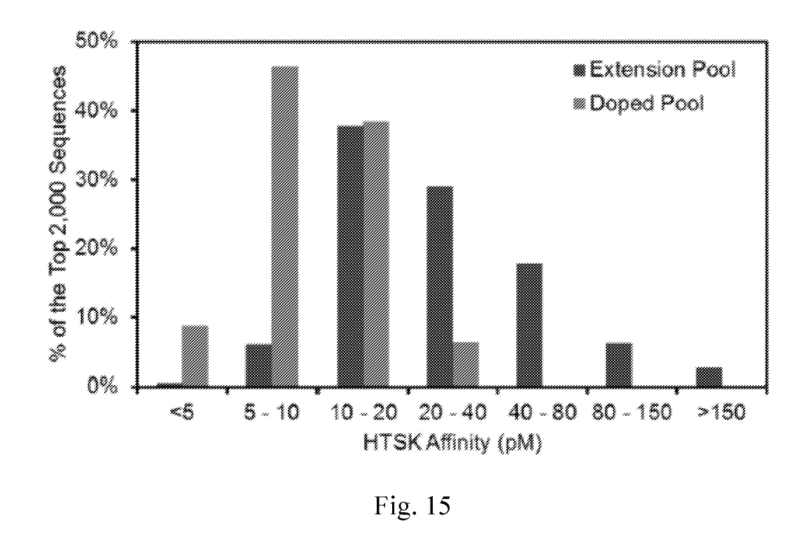

Based on the HTSK results, peptide D79 (frequency rank of 79 in the doped selection pool) is identified with a k_{off} value of 5.9×10⁻⁷, which is over three times slower than the previously identified slowest off-rate peptide ligand (D1) or the biotin-streptavidin interaction (FIG. 13d). In addition, peptide E1452 (frequency rank of 1452 from the extension selection pool) is identified with the k_{off} value of 8.5×10⁻⁷, which is over two fold slower than D1 (FIG. 14). These results show the ability of the extension selection pool to generate ultra-high affinity ligands without the need for a biased (doped) selection pool to improve affinity further. The HTSK method was used to identify thousands of sequences at a modest chain length (21 amino acids long) which have a 10 pM K_{d} or better (FIGS. 14 and 15). Thus, the present method is suitable for high affinity fusion ligands (K_{d}<10 nM) since the slower off-rates allow for more precise measurements. The above results show that the HTSK method is reproducible and accurate, and have identified the highest affinity peptide-protein interaction yet discovered.

For reasons of completeness, various aspects of the invention are set out in the following numbered clauses:

Clause 1. A method for simultaneously determining [L]₀ and K_{d} of a ligand for a target protein, the method comprising:
(1) conducting a first quantitative equilibrium immunoassay of the ligand with the target protein at a first concentration of the target protein;
(2) conducting a second quantitative equilibrium immunoassay of the ligand with the target protein at a second concentration of the target protein; and
(3) fitting the data resulting from steps (1) and (2) to determine K_{d} and [L]₀ simultaneously.

Claus 2. The method of clause 1, wherein the ligand is selected from the group consisting of an antibody, a peptide, and a small molecule compound.

Clause 3. The method of clause 2, wherein the ligand is selected from the group consisting of an antibody and a peptide.

Clause 4. The method of clause 2, wherein the concentration of the ligand is unknown.

Clause 5. The method of clause 3, wherein the ligand is immobilized and the target protein is in solution.

Clause 6. The method of clause 3, wherein the target protein is immobilized and the ligand is in solution.

Clause 7. The method of clause 1, wherein the target protein is B-cell Lymphoma extra-large protein (Bcl-xL).

Clause 8. The method of clause 7, wherein the ligand is a monoclonal antibody.

Clause 9. The method of clause 1, wherein the quantitative equilibrium immunoassay comprises incubating the ligand and the target protein to equilibrium.

Clause 10. The method of clause 1, wherein the quantitative equilibrium immunoassay is an Enzyme-linked Immunosorbent Assay (ELISA).

Clause 11. The method of clause 1, wherein the quantitative equilibrium immunoassay is an Acoustic Membrane MicroParticle (AMMP) assay.

Clause 12. The method of clause 1, wherein the fitting of step (3) comprises a monovalent model for the binding between the target protein and the ligand.

Clause 13. The method of clause 12, wherein the monovalent model is

$${[ C ]}_{EQ} = \frac{{[ T ]}_{0} + {[ L ]}_{0} + {K}_{D} - \sqrt{{( {[ T ]}_{0} + {[ L ]}_{0} + {K}_{D} )}^{2} - {{4 [ T ]}_{0} [ L ]}_{0}}}{2}$$

wherein

[C]_{EQ} represents the concentration of the target-ligand complex at equilibrium;

[T]₀ represents the initial concentration of the target protein; and

[L]₀ represents the initial concentration of the ligand.

Clause 14. The method of clause 1, wherein the fitting of step (3) comprises a divalent model for the binding between the target protein and the ligand.

Clause 15. The method of clause 14, wherein the divalent model is

$${[ TL ]}_{EQ}^{3} ( - 4 {K}_{d 1} + {K}_{d 2} ) + {[ TL ]}_{EQ}^{2} ( - 4 {K}_{d 2} {K}_{d 1} + {K}_{d 2}^{2} - 2 {{K}_{d 2} [ L ]}_{0} ) + {[ TL ]}_{EQ} ( 2 {{{K}_{d 2} [ T ]}_{0} [ L ]}_{0} - {K}_{d 2}^{2} ( {K}_{d 1} + {[ T ]}_{0} + {[ L ]}_{0} ) - {{K}_{d 2} [ T ]}_{0}^{2} ) + {{[ T ]}_{0} [ L ]}_{0} {K}_{d 2}^{2} = {0 \\ [ {T}_{2} L ]}_{EQ} = \frac{{{[ T ]}_{0} [ TL ]}_{EQ} - {[ TL ]}_{EQ}^{2}}{{K}_{d 2} + {2 [ TL ]}_{EQ}}$$

wherein

[T]₀ represents the initial concentration of the target protein;

[L]₀ represents the initial concentration of the ligand;

[TL]_{EQ} represents the concentration of a monovalently bound target-ligand complex TL at equilibrium, in which the molar ratio of the target protein to the ligand is 1:1;

[T₂L]_{EQ} represents the concentration of a divalently bound target-ligand complex T₂L at equilibrium, in which the molar ratio of the target protein to the ligand is 2:1;

K_{d1} represents the dissociation constant in the binding of the ligand to the target protein to form the monovalently bound target-ligand complex TL; and

K_{d2} represents the dissociation constant in the binding of the monovalently bound target-ligand complex TL to the target protein to form the divalently bound target-ligand complex T₂L.

Clause 16. The method of clause 1, wherein the quantitative equilibrium immunoassay is a quantitative equilibrium exclusion immunoassay.

Clause 17. A method for determining binding affinity, the method comprising:
(1) preparing a pool of candidate ligands;
(2) mixing the pool of candidate ligands with a target protein immobilized on a carrier;
(3) isolating the mixture of step (2);
(4) sequencing the candidate ligands bound to the target protein to identify a pool of nucleic acid sequences;
(5) translating each of the nucleic acid sequences in the pool of sequences identified in step (4); and
(6) calculating a frequency of each translated sequence generated in step (5).

Clause 18. The method of clause 17, wherein each of the candidate ligands is selected from the group consisting of a fusion ligand in which a nucleic acid is fused to a protein, a peptide, or a small molecule, an mRNA, a DNA, and an nucleic acid aptamer.

Clause 19. The method of clause 17, wherein the pool of candidate ligands comprises mRNA-peptide fusion molecules.

Clause 20. The method of clause 17, wherein the isolating in step (3) is carried out at a series of predetermined time points.

Clause 21. The method of clause 17, further comprising
(7) calculating a fractional composition of each translated sequence generated in step (5); wherein the fractional composition of a translated sequence is the frequency of the sequence obtained in step (6) divided by the total sequences in the pool.

Clause 22. The method of clause 17, further comprising calculating the kinetic on-rate for a ligand molecule identified in step (4).

Clause 23. The method of clause 17, further comprising calculating the kinetic off-rate for a ligand molecule identified in step (4).

Clause 24. The method of clause 17, wherein the target protein is B-cell lymphoma extra-large protein (Bcl-xL).

Clause 25. The method of clause 17, wherein the carrier comprises magnetic beads.

Clause 26. The method of clause 17, wherein the sequencing in step (3) comprises next-generation sequencing.

Clause 27. The method of clause 17, further comprising calculating a K_{d} value for a ligand molecule identified in step (4).

Various features and advantages of the invention are set forth in the following claims.

What is claimed is:

1. A method for simultaneously determining [L]₀ and K_{d} of a ligand for a target protein, the method comprising:
(1) conducting a first quantitative equilibrium immunoassay of the ligand with the target protein at a first concentration of the target protein;
(2) conducting a second quantitative equilibrium immunoassay of the ligand with the target protein at a second concentration of the target protein; and
(3) fitting the data resulting from steps (1) and (2) to determine K_{d} and [L]₀ simultaneously.

2. The method of claim 1, wherein the ligand is selected from the group consisting of an antibody, a peptide, and a small molecule compound.

3. The method of claim 2, wherein the ligand is selected from the group consisting of an antibody and a peptide.

4. The method of claim 2, wherein the concentration of the ligand is unknown.

5. The method of claim 3, wherein the ligand is immobilized and the target protein is in solution.

6. The method of claim 3, wherein the target protein is immobilized and the ligand is in solution.

7. The method of claim 1, wherein the target protein is B-cell Lymphoma extra-large protein (Bcl-xL).

8. The method of claim 7, wherein the ligand is a monoclonal antibody.

9. The method of claim 1, wherein the quantitative equilibrium immunoassay comprises incubating the ligand and the target protein to equilibrium.

10. The method of claim 1, wherein the quantitative equilibrium immunoassay is an Enzyme-linked Immunosorbent Assay (ELISA).

11. The method of claim 1, wherein the quantitative equilibrium immunoassay is an Acoustic Membrane MicroParticle (AMMP) assay.

12. The method of claim 1, wherein the fitting of step (3) comprises a monovalent model for the binding between the target protein and the ligand.

13. The method of claim 12, wherein the monovalent model is
$${[ C ]}_{EQ} = \frac{{[ T ]}_{0} + {[ L ]}_{0} + {K}_{D} - \sqrt{{( {[ T ]}_{0} + {[ L ]}_{0} + {K}_{D} )}^{2} - {{4 [ T ]}_{0} [ L ]}_{0}}}{2}$$
wherein
[C]_{EQ} represents the concentration of the target-ligand complex at equilibrium;
[T]₀ represents the initial concentration of the target protein; and
[L]₀ represents the initial concentration of the ligand.

14. The method of claim 1, wherein the fitting of step (3) comprises a divalent model for the binding between the target protein and the ligand.

15. The method of claim 14, wherein the divalent model is
$${[ TL ]}_{EQ}^{3} ( - 4 {K}_{d 1} + {K}_{d 2} ) + {[ TL ]}_{EQ}^{2} ( - 4 {K}_{d 2} {K}_{d 1} + {K}_{d 2}^{2} - 2 {{K}_{d 2} [ L ]}_{0} ) + {[ TL ]}_{EQ} ( 2 {{{K}_{d 2} [ T ]}_{0} [ L ]}_{0} - {K}_{d 2}^{2} ( {K}_{d 1} + {[ T ]}_{0} + {[ L ]}_{0} ) - {{K}_{d 2} [ T ]}_{0}^{2} ) + {{[ T ]}_{0} [ L ]}_{0} {K}_{d 2}^{2} = {0 \\ [ {T}_{2} L ]}_{EQ} = \frac{{{[ T ]}_{0} [ TL ]}_{EQ} - {[ TL ]}_{EQ}^{2}}{{K}_{d 2} + {2 [ TL ]}_{EQ}}$$
wherein
[T]₀ represents the initial concentration of the target protein;
[L]₀ represents the initial concentration of the ligand;
[TL]_{EQ} represents the concentration of a monovalently bound target-ligand complex TL at equilibrium, in which the molar ratio of the target protein to the ligand is 1:1;
[T₂L]_{EQ} represents the concentration of a divalently bound target-ligand complex T₂L at equilibrium, in which the molar ratio of the target protein to the ligand is 2:1;
K_{d1} represents the dissociation constant in the binding of the ligand to the target protein to form the monovalently bound target-ligand complex TL; and
K_{d2} represents the dissociation constant in the binding of the monovalently bound target-ligand complex TL to the target protein to form the divalently bound target-ligand complex T₂L.

16. The method of claim 1, wherein the quantitative equilibrium immunoassay is a quantitative equilibrium exclusion immunoassay.

17. A method for determining binding affinity, the method comprising:
(1) preparing a pool of candidate ligands;
(2) mixing the pool of candidate ligands with a target protein immobilized on a carrier;
(3) isolating the mixture of step (2);
(4) sequencing the candidate ligands bound to the target protein to identify a pool of nucleic acid sequences;
(5) translating each of the nucleic acid sequences in the pool of sequences identified in step (4); and
(6) calculating a frequency of each translated sequence generated in step (5).

18. The method of claim 17, wherein each of the candidate ligands is selected from the group consisting of a fusion ligand in which a nucleic acid is fused to a protein, a peptide, or a small molecule, an mRNA, a DNA, and an nucleic acid aptamer.

19. The method of claim 17, wherein the pool of candidate ligands comprises mRNA-peptide fusion molecules.

20. The method of claim 17, wherein the isolating in step (3) is carried out at a series of predetermined time points.

21. The method of claim 17, further comprising
(7) calculating a fractional composition of each translated sequence generated in step (5); wherein the fractional composition of a translated sequence is the frequency of the sequence obtained in step (6) divided by the total sequences in the pool.

22. The method of claim 17, further comprising calculating the kinetic on-rate for a ligand molecule identified in step (4).

23. The method of claim 17, further comprising calculating the kinetic off-rate for a ligand molecule identified in step (4).

24. The method of claim 17, wherein the target protein is B-cell lymphoma extra-large protein (Bcl-xL).

25. The method of claim 17, wherein the carrier comprises magnetic beads.

26. The method of claim 17, wherein the sequencing in step (3) comprises next-generation sequencing.

27. The method of claim 17, further comprising calculating a K_{d} value for a ligand molecule identified in step (4).


SEQUENCE LISTING (9 sequences)

SEQ ID NO: 1 - protein, 19 aa, Artificial Sequence
  N-terminal avitag
AGGLNDIFEAQKIEWHEGG

SEQ ID NO: 2 - protein, 24 aa, Artificial Sequence
  Peptide E1
MIETITIYNYKKAADHFSMSMGSK

SEQ ID NO: 3 - protein, 24 aa, Artificial Sequence
  Peptide E2
MIETITIYKYKKAADHFSMSMGSK

SEQ ID NO: 4 - protein, 24 aa, Artificial Sequence
  Peptide D1
MIAISTIYNYKKAADHYAMTKGSK

SEQ ID NO: 5 - protein, 29 aa, Artificial Sequence
  Peptide Bim
MDMRPEIWIAQELRRIGDEFNAYYARRGK

SEQ ID NO: 6 - protein, 24 aa, Artificial Sequence
  Peptide D79
MIDTNVILNYKKAADHFSITMGSK

SEQ ID NO: 7 - protein, 21 aa, Artificial Sequence
  Peptide E1 clone
MIETITIYNYKKAADHFSMSM

SEQ ID NO: 8 - protein, 21 aa, Artificial Sequence
  Peptide D1 clone
MIAISTIYNYKKAADHYAMTK

SEQ ID NO: 9 - protein, 21 aa, Artificial Sequence
  Peptide D79 clone
MIDTNVILNYKKAADHFSITM